(19) United States
(12) Patent Application Publication
(10) Pub. No.: US 2021/0304155 A1
(43) Pub. Date: Sep. 30, 2021

(54) SYSTEM FOR VERIFICATION AND MANAGEMENT OF PAIRED ASSETS ASSOCIATED WITH A STRUCTURE

(71) Applicant: Scientia Potentia Est., LLC, Charleston, SC (US)

(72) Inventors: Jeremy Blackburn, Charleston, SC (US); Justin Southward, Lehigh Acres, FL (US); W. Kurt Taylor, N. Charleston, SC (US); Karl David, Charleston, SC (US); Austi Critchfield, Clearwater, FL (US); Michael Lu, N. Charleston, SC (US); Tim McVicker, Charleston, SC (US)

(73) Assignee: Scientia Potentia Est., LLC, Charleston, SC (US)

(21) Appl. No.: 17/344,043

(22) Filed: Jun. 10, 2021

Related U.S. Application Data
(63) Continuation-in-part of application No. 17/176,056, filed on Feb. 15, 2021.
Application No. 17/176,056 is a continuation-in-part of application No. 17/128,084, filed on Dec. 19, 2020.
Application No. 17/128,084 is a continuation-in-part of application No. 16/997,840, filed on Aug. 19, 2020.
Application No. 16/997,840 is a continuation-in-part of application No. 16/994,585, filed on Aug. 15, 2020.
Application No. 16/994,585 is a continuation-in-part of application No. 16/991,916, filed on Aug. 12, 2020.
Application No. 16/991,916 is a continuation-in-part of application No. 16/876,080, filed on May 17, 2020.
Application No. 16/876,080 is a continuation-in-part of application No. 16/810,782, filed on Mar. 5, 2020.
Application No. 16/810,782 is a continuation-in-part of application No. 16/510,634, filed on Jul. 12, 2019, now Pat. No. 10,713,737.
Application No. 16/510,634 is a continuation-in-part of application No. 16/452,076, filed on Jun. 25, 2019.
Application No. 16/810,782 is a continuation-in-part of application No. 16/510,634, filed on Jul. 12, 2019, now Pat. No. 10,713,737.
Application No. 16/510,634 is a continuation-in-part of application No. 16/452,076, filed on Jun. 25, 2019.
Application No. 16/810,782 is a continuation-in-part of application No. 16/452,076, filed on Jun. 25, 2019.

(51) Int. Cl. G06Q 10/00; G06Q 20/10; G06Q 20/02; G06Q 20/38; G06Q 50/06; G06Q 30/00; G06Q 10/08

(52) U.S. Cl. CPC G06Q 10/20; G06Q 20/102; G06Q 20/027; G06Q 30/018; G06Q 50/06; G06Q 30/012; G06Q 10/0875; G06Q 20/389

(57) ABSTRACT

A computerized system for pairing physical objects with virtual representations comprising: a structure having a static location; a controller uniquely associated with the structure and in communication with an immutable ledger, a remote delivery computer system, a payment management computer system and a remote maintenance computer device; an input assembly in communications with the controller; a set of computer readable instructions included in the controller adapted for management of a payment account selected from the group consisting of a mortgage, lease, rent, utility, insurance, tax, common area dues, homeowner dues, regime fees, and any combination thereof and managing a maintenance request.

### RELATED APPLICATIONS

This application is a continuation in part of U.S. application Ser. No. 17/230,911 filed Apr. 14, 2021 which is a continuation in part of U.S. patent application Ser. No. 17/128,084 filed Dec. 19, 2020 which is a continuation in part of U.S. application Ser. No. 16/997,840 filed Aug. 19, 2020, which is a continuation in part of U.S. patent application Ser. No. 16/994,585 filed Aug. 15, 2020 entitled “System For Management Of Verification Of Project Commencement and Completion”, which in turn is a continuation in part of U.S. patent application Ser. No. 16/991,916 entitled “System For Management Of Warranty Information For Projects And Materials”, filed on Aug. 12, 2020 which in turn is a continuation in part of U.S. patent application Ser. No. 16/876,080 entitled “Digital Asset System For Management Of Projects And Materials”, filed May 17, 2020 which in turn is a continuation in part of U.S. patent application Ser. No. 16/810,782, entitled “System For Management And Verification of Code Compliance”, filed on Mar. 5, 2020 which in turn is a continuation in part of U.S. patent application Ser. No. 16/410,634, entitled “Use of an Immutable ledger Reference Construction Metadata and to Use Smart Contracts for a Project or process”, filed on Jul. 12, 2019, U.S. patent application Ser. No. 16/510,642 entitled “Use of a Blockchain-Based Distributed Ledger and Smart Contracts for a Project or process”, filed on Jul. 12, 2019 both of which are continuations of U.S. patent application Ser. No. 16/452,076, entitled “Site Super System For Structures”, filed Jun. 25, 2019 all of which are incorporated reference.

### BACKGROUND

### 1) Field of the System

A system for pairing physical assets, transaction, structure and occupant to verifiably link the physical asset, transaction, structure and occupant with respective virtual representation to create a undichotomized pair providing truth over trust.

### 2) Background

In the modern economy, there is a continuing trend for digitization. This trend includes attempts to create digital assets and digital representations that attempt to represent physical assets. Through digitization, an asset, transaction, structure and individual can be converted into a digital representation stored on a database. For example, a bank balance can be the digital representation of the fiat currency that is in the possession of the bank account holder. The challenge is when the asset is not fungible, such as currency, but are not easily substituted. For example, during the manufacturing of a vehicle, components for that vehicle are specific to that make, model and year and are not readily substituted between vehicles of different makes, models, and years. This can be illustrated when replacement parts are needed for the vehicle and the make, model, and year, are need for determining the proper replacement and in some cases the vehicle identification number (VIN) number itself is needed because the replacement part is unique to the that vehicle. An additional problem is created when a first replacement is placed on a vehicle and a second part that cooperates with the first part needs to be replaced. The second replacement part, while compatible with the original part, may not be compatible with the first replacement part. Currently, there is no digital system that allows for the tracking of physical assets and the processes that use the physical asset (e.g., installation) using a virtual representation of the physical asset.

In the financial industry, digitization began with electronic information representing the dollar value of an account. While the value of the account was not tied to a physical dollar, it did represent the number of dollars in the account. As the financial industry progressed, the electronic current itself became the asset as discussed in U.S. Pat. No. 9,135,787, this patent discloses a Bitcoin kiosk/ATM that facilitates the buying or selling of Bitcoin. For example, Bitcoin can be traded so that Bitcoin is in and of itself the commodity and does not necessarily represent a fiat currency.

This type of digitization where the digital information represents the asset that is to be distinguished with electronic scanning of physical assets. For example, digital information representing US currency is quite different than physically scanning US currency. Despite the illegality of this example, the digital scan of US currency and the US currency itself are not equivalent. The US currency can be spent without reference or modification of the digital scan and the digital scan can be manipulated without reference or modifications to the US currency. The physical and the digital are not verifiably paired. This distinction makes the digitization of physical assets challenging as the digital asset and the virtual representation are not functional equivalents and therefore are not verifiably paired. While it is a fairly simple task to put a digital item on digital immutable ledger, such as blockchain, it is difficult, if not impossible, to put a physical item on digital media.

In the current systems, especially with digital currencies, there is a significant risk of scams, frauds, and irregularities with the assets. Potential risks in this area include Ponzi schemes, fake initial coin offering and fraudulent exchanges. It would be advantageous to have a system that can reduce or eliminate the risks associated with cryptocurrency as well as digital representation of physical assets.

A significant disadvantage with current systems is the potential for rehypothecation. First, hypothecation means pleasing an asset as collateral for a debt so that in the event of a default, the asset can be seized (e.g., foreclosure or repossession) to satisfy the default at least partially on the debt. Rehypothecation is when the creditor uses the collateral from a first loan (e.g., original loan) and uses it as collateral for a second loan. Rehypothecation increases uncertainty and adds risk in that actual ownership, lien, or collateral can become uncertain.

For many industries, there should be a systematic and logical workflow that increases the success of any project, manufacturing, construction, providing of services, and the like. For example, when constructing a residential home, the foundation is provided prior to the roof installation. The design record can include make, model, serial number, or other identification of for every component in the task, project, or process. The design record may include workers qualified to perform and complete the work, who should inspect the work and how the project should be insured and funded.

This process can require that the designer specify materials, suppliers that supply the materials, that workers have a specific set of credentials, licenses or experience, and inspectors verify the delivery and performance of tasks during the project. When designing a project such as manufacturing, repairs, construction, maintenance, testing, and the like, designers can include a material list (e.g., bill of materials, parts list, packing list) representing the specified materials to be used for the project. For the project to be successful, the specified materials should be delivered to the project site and verified as correct. The workers should be properly trained and equipped according to the materials and associated tasks. Third-party inspections should be performed for each step in the process to verify compliance with regulations, specifications, instructions and the like.

In current workflow and material tracking processes, one disadvantage is the lack of accountability, verification and reliability of information related to the materials used, tasks performed and workers. The inability to verify correct materials, installation, workers, coworkers' experience and credentials and other factors can result in loss, mistakes, injury, increased insurance claims and increased premiums. This weakness in the current process and system negatively impact the process, risks, and costs in general. While there have been some attempts to add item information to a physical material, such as U.S. Pat. No. 8,321,302, these attempts have focused on tracking inventory levels and do not include verifiably pairing a physical material with a virtual representation that can be tracked throughout a process. Further, these prior attempts focus on a single location and do not consider the fact that the design, material, and project can initially be at separate locations. This disadvantage can be seen in U.S. Pat. No. 8,521,620 which specifically states that if a RFID tag is lost or damaged, the system allows a user to enter an item number or style and tags of similar items are displayed, a new tag is generated and associated with the item having the lost or damaged tag. Once the RFID tag is lost or damaged, the physical asset is no longer paired. While this system specifically allows for the replacement of RFID tags on the same item, it lacks the ability to verifiably pair the new tag with the physical asset.

The disadvantages of current systems are caused in part due to the lack of pairing the physical asset with the virtual representation to determine that the materials requested, and the materials delivered and used are properly paired. Attempts to provide for inspections that the proper materials designated at the design were actually delivered and used or installed at a target location. United States Patent Application Publication 2019/0287181 discloses a virtual home inspection but specifically states that the inspector does not have to be tied to the physical location where the home is to be inspected. Without the ability to verify that the materials inspected are physically located at the structures, a virtual inspection has all the disadvantages of mistakes, inaccuracies, and even dishonestly with designated materials not arriving and being used or installed at the home.

There is also a need to verify that the workers preforming tasks associated with the materials are properly licensed, experienced, and authorized to perform these tasks. Laws and regulations require that many tasks performed by workers must be performed by a properly licensed worker. Licensing is intended to ensure that only competent and ethical individuals practice in an occupation and include professions such as land surveyors, cosmetologists, nurses, building contractors, engineers, and electricians. Failure to use licensed workers can lead to poor performance, low quality, fines, legal liability and even criminal liability. There have been attempts to manage worker licensing such as shown in U.S. Pat. No. 8,103,596 but such attempts fail to determine if the task actually being performed by the worker is one that the worker is licensed to perform. These prior attempts focus on maintaining licensing requirements, but do not verify that the worker is actually licensed to perform that task.

There is also a need to verify that the tasks being performed by the worker using verified materials is in compliance with the applicable specifications. Specifications in some processes can include the specification of a manufacturer or project site, the performance criteria of the materials, worker experience requirements and the quality of the systems and products, which standards are applicable and how they should be executed, who should execute them and even the specific model of a product to use. Specifications can include materials selected at the design phase as well as handling and installation specifications associated with materials from the supplier. It would be advantageous to have a system that verified proper materials, authorized workers are used and proper installation procedures under the proper environmental conditions are performed and pairing these items and tasks with a virtual representation. It would be advantageous to have a system that reduced or eliminate the risk of a substitute, lesser quality or other non-designated or approved material being used. It would be advantageous for a system that prevented or reduced the risk of unlicensed or unauthorized workers, failures to comply with material specifications and failure to follow installation or user instructions. It would be advantageous to have a system that provides for third party or automated independent verification to reduce false or misleading information, incorrect signoffs on a project, incorrect inspections and improper material and tasks in general.

The use of inspectors can assist with reducing the risk of improper or unauthorized workers, materials, tasks, process, and specifications. One disadvantage of an inspection is that it occurs at a point in time and cannot verify proper workers, materials, tasks and use and installation specifications. For example, in the construction of vehicles, electronics, resident and commercial buildings and other articles, wiring, to be properly inspected, needs to be reviewed before it is obscured. For example, during the residential construction project, if the interior walls are installed, the material and installation behind the walls cannot be inspected. There have been attempts to provide for automated inspection such as U.S. Pat. No. 7,508,973 which discloses method of inspecting detects includes assigning a plurality of sets of image acquisition conditions, executing inspection using each of the sets of conditions, classifying all detected defects into real defects and false defects by use of an automatic defect classification function, and selecting, from the plurality of sets of conditions, a set of conditions ideal for detection. However, this attempt does not allow the inspector to verify that the material used and processed complies with the design and specification using a paired virtual representation.

Another disadvantage with the current system is that the data resulting from the materials, workers, tasks, and the like are not easily accessible by all stakeholders. For example, inspections can be a physical form or captured with a proprietary inspection system, Inspection Support Network®. The data is stored and maintained by the system provider so that data security is dependent upon the accuracy of the input and the system provider itself. It would be advantageous to have a permanent verified record of a design, materials, delivery, workers, tasks, installation, and inspections paired with physical materials and activities that can be referenced by others before, during and after commencement of a project or process.

There have been some attempts to improve tracking of articles such as shown in U.S. Pat. No. 7,898,403 that are directed to a method and system for detecting construction equipment process failures. A database is populated from information from a third-party source and a process failure report is provided for processes that are outside a norm assigned to the construction equipment asset. U.S. Pat. No. 7,031,930 is directed to a method and system for managing complex projects or processes by monitoring subcontractors in real time, against a system after commencement of the project. U.S. Pat. No. 8,004,397 is directed to a mountable reporting source comprising a controller coupled with an interrogating component configured for automatically receiving an identifier which is unique to an asset having a position determining component. Again, this is directed to the construction process itself, not management and verification of code compliance. U.S. Pat. No. 8,428,904 discloses product integrity tracking system, shipping label and associated method. This patent is directed to label body for attaching to a product to be shipped or to packaging containing the product. It does not disclose a verifiable pairing between the physical asset and the virtual representation during the project's lifetime.

Further, prior systems do not account for delivery of materials in undesirable weather conditions. Materials that are exposed to undesirable weather can damage or destroy the material making it unfit for use. It would be advantageous for a system to determine the environment where materials are delivered and installed to prevent damage to the material and therefore the project. For example, when moisture gets trapped behind the building material stucco, the moisture can produce “stucco tears,” which result in discoloration of the stucco beneath windows or cause the formation of mold and mildew, which can escalate into an infestation of black mold. Manufacturers have certain requirements for stucco and its installation and can require certain humidity ranges and temperature ranges. If metal, especially carbon steel, is exposed to moisture, chloride, and other environmental materials, the steel can corrode such as when metal like steel, copper, magnesium, aluminum is exposed to free flowing air and moisture.

It would be an advantage to have a system that can pair physical assets with virtual representation so that compliance with the design, process or procedure, and specifications are verified and stored as a persistent record.

It would be advantageous to have a system that provides for multiparty verification of the pairing of a physical asset with a virtual representation for tracking of the physical asset and the associated project.

It would be advantageous to have a system that provides for a verified trustworthy association between physical material and virtual representations.

### SUMMARY OF THE SYSTEM

In accordance with an exemplary embodiment,

The metadata can be taken from the group consisting of date, time, location, supplier, environmental condition, and any combination thereof.

### BRIEF DESCRIPTION OF THE DRAWINGS

FIGS. 1A-1B shows various side views of aspects of the system.

FIG. 1C shows an exemplary floor plan.

FIG. 2A is a block diagram of aspects of the system.

FIG. 2B shows aspects of a user interface.

FIG. 2C shows a flowchart of aspects of the system.

FIG. 3A shows an example of a communications environment.

FIG. 3B shows an example of an immutable ledger.

FIG. 3C shows a schematic of the aspects of the system.

FIG. 3D shows a schematic of the aspects of the system.

FIG. 4 shows a flowchart illustrating aspects of the system.

FIG. 5 shows a flowchart illustrating aspects of the system.

FIG. 6 shows various types of biometric data that may be gathered.

FIG. 7 shows types of events that may be triggered.

FIG. 8 shows a flowchart illustrating aspects of the system.

FIG. 9 shows a flowchart illustrating aspects of the system.

FIG. 10 shows a flowchart illustrating aspects of the system.

FIG. 11 shows a flowchart illustrating aspects of the system.

FIG. 12A shows a plan view of a structure.

FIG. 12B shows geofencing areas at a structure.

FIG. 13 shows flowchart having steps that may be performed.

FIG. 14 carious sensors in the input assembly.

FIG. 15 shows flowchart having steps that may be performed.

FIG. 16 shows a flowchart illustrating steps that may be performed.

FIG. 17 shows a flowchart illustrating steps that may be performed.

FIG. 18 shows a flowchart illustrating steps that may be performed.

FIG. 19 shows informational flow of the system.

FIG. 20 shows flowcharts of operations performed by system.

FIG. 21 shows flowcharts of operations performed by system.

FIG. 22 shows flowcharts of operations performed by system.

FIG. 23 shows examples smart contracts.

FIG. 24 shows flowcharts of steps performed by the system.

FIGS. 25A-25D shows schematics of aspects of the system.

FIGS. 26A-26C shows schematics of aspects of the system.

### DETAILED DESCRIPTION

The present system provides for a system for verification and management of paired assets associated with a structure that can includes real time verified pairing of a physical item, structure, individual and transactions with respective virtual representations. The server can include a set of server computer readable instructions configured to receive a component (i.e., material) history and acceptable replacement part that can include fields as make, model, quantity, warrant inflation, hazardous information, safety data, the class, type or other identification of the component or part as well as, one or more vendors, the cost, installation instructions, material specifications, assembly, other information and material. The component history or replacement part may contain a complete itemization of associated components needed for a task, project, process or assembly. The component information (e.g., component history or replacement part) may be updated as actual materials arrive to a structure and have serial numbers, bar codes, QR codes, RFID values, beacons, lots, sizes, or other components identification added or associated with the component information.

For example, the component information can include information about the component as well as information as the structure where the component is designed to be installed or re-installed in the case of a replacement part. The structure can include a location marker that uniquely associated the structure with a physical location. The location can be associated with a construction site when the structure was completed.

The system can also provide component information to a supply computer device informing a third-party supplier, distributor or reseller to deliver the component to the structure. The supplier can review the component request and determine if the component specified is available, in stock, consistent other components requested or otherwise determine information concerning the ability to provide the specified component. The supplier can receive the component request from the system or can retrieve the component request from the immutable ledger. The component request can be included in a maintenance request that is associated with the structure or the occupant.

The supply company can send proposed modifications to the component request which can be accepted or rejected by the requesting party such as a maintenance worker or occupant of the structure. Using information from a supplier, the component information can include a virtual representation of the component. In one embodiment, the component can be specified by class, type, code, number of other identifying information and virtual representation so that the component information includes the component paired with the virtual representation which is verified by the system, a remote computer device or an individual. This verification can be independent of the other verifications described herein.

The structure can include a structure history which can include a building information model for a construction project associated with the structure, a listing of repair components and parts, a listing of material from the original construction, and other applications where components are selected, ordered, shipped, and used. A supply record can be created which can include component information and the virtual representation. The supply company can inspect the component to ensure that the component is properly associated with the virtual representation to create a verified pairing of the component with the virtual representation. The suppler record, that can include the verified paired information, can be stored on the immutable ledger.

When the component is selected for transport by the supplier, a shipping company can be sent a shipping order representing the materials to be transported. The shipping order can include a listing of the materials and the verifiably paired virtual information. The shipping company can be provided shipping information that can facilitate the transaction (e.g., broker, distributer, reseller). The shipping order can be provided directly to the shipping company or can be retrieved from the immutable ledger. Once the shipping company receives the shipping order, it can travel to the component location and determine if the list of components in the shipping order match the physical components at the component location. A shipping pickup record can be created representing that the shipping company received the component(s) and that the component(s) picked match the shipping order.

Once that component is delivered to the structure, the shipping company can delivery the component and verify that the component delivered is the same component included in the shipping order. The shipping company can create a shipping delivery record representing what component was delivered and that the component matches the shipping order. The shipping record can include the environmental conditions when the component was delivered, delivery notes and the like. The shipping delivery record can be stored on the immutable ledger. A worker including the occupant, can install the component or replace a component associated with the structure. The worker can review the component delivered and verify that the delivered component matches the maintenance request, component requirement, supply record, shipping order, shipping pickup record, shipping delivery record of any combination.

Once the component is received at the structure, the system can notify workers that the component is ready for use. The worker can be verified by the system and a worker verification record which is created and can be stored on the immutable ledger. The system can utilize biometrics or other systems as described herein for verification of actual workers at the structure and can verify that the worker has the proper licenses, work certifications, experience, and authorizations as well as for verified payroll, warranty and insurance coverage. The system can identify workers, occupants, owners and other individuals entering or leaving the structure and store this information on the immutable ledger. The verification can be through identification devices such as a camera or other image capture device, facial recognition, voice recognition, retinal scans, fingerprint scanners, hand scanners, and other devices including biometric devices. In one embodiment, the system may allow authorized individuals to manually enter the presence of another authorized individual using an input assembly at the structure or through a remote device that can be determined to be at the structure location, within a boundary associated with the structure, and in proximity to the structure. In one embodiment, individuals may be verified and paired with a virtual representation using two-factor authentication.

The worker, occupant and owner can be provided with component installation information and specifications or other requirements that can be represented by a maintenance request. The maintenance request can be stored on the immutable ledger. Once the component is installed, the system can verify that the component was installed according to the maintenance request, create a maintenance record, and store the maintenance record on the immutable ledger. The maintenance record represents that a task associated with the component was properly completed. The maintenance request can represent that the task was performed by proper worker, with the proper component, using the proper process, in compliance with a regulatory code, in compliance with specifications, pass one or more inspections.

Prior to, during and after an installation or other activity is completed, an inspection can be performed that can include a pre-installation inspection, task inspection, post-installation inspection and any combination. A pre-installation inspection, installation inspection, and post installation inspection record can be created so that these records can be stored on the immutable ledger. The maintenance request can include information that the inspection resulted in passing, passing with deficiencies, and failing. In the event that the inspection fails, the worker can be given the opportunity to remedy the failure and the inspection process can be performed again. The work can also determine if, while the installation or repair passed the inspection, the deficiencies should be remedied.

The system can be uniquely associated with the structure. A structure marker can be affixed to the structure or structure location. The location marker can be read by the system so that the system can determine its own location. Third parties can read the location marker to determine the location of the structure and the third party when present at the structure. For example, the shipping company can arrive at the structure, read the location marker, and associate the location marker with the delivery of an article such as a component. In one embodiment, the worker, occupant, or owner can receive a shipping identifier associated with the delivery, such as a truck, trailer, pallet, or other container so that the article is known to be received at the structure. Other parties can also access the location marker to verify that the third party is at the structure.

The system can be contained in a housing such as a kiosk and can be physically associated with the structure. The structure can be defined by a boundary representing the perimeter of the structure location. The system can include a sensor and reader which can be selected from the group consisting of radio frequency identification (RFID) detector, ultra-high frequency (UHF) detector, a bar code scanner, a QR code scanner, near frequency communication (NFC) device; Bluetooth beacons, an optical character recognition (OCR) device and any combination thereof. An environmental sensor, such as a weather sensor or weather station, can be in communications with the or included in the system and configured to record the weather and other environmental conditions at the structure location and at different times during an activity, project, transaction and the like.

The system may record the date and time of events such as the arrival and departure of materials, components, packages, deliveries, individuals, workers, supplies, third parties, inspections, and the like to and from the structure, the date and time associated with environmental conditions including weather. Additionally, the system may prevent tasks, such as repairs, from being performed if the tasks would violate manufacturer, supplier, integrator, customer, or other guidelines, specifications or instructions for use or installation of a component. Recording environmental information, including weather, at the structure allows for autonomous confirmation of environmental conditions that do not rely solely on third party sources or sources that are general or distant from the structure location.

The system may also determine if an unidentified individual attempts to enter the structure, the system may take the appropriate responses, such as sending notifications, triggering alarms, and/or contacting law enforcement authorities or security. The decision as to the appropriate response may be determined by, the date, the time, current weather conditions, authorizations, project or process status, or related factors.

The smart locks may also be used to limit access to certain portions of the structure. For example, a utility area may be included in the structure that only has access through a certain entry. An individual's right to access this area may be dictated by permissions that are stored through each party involved or associated with the structure. A maintenance worker may be allowed access to the utility area without being granted access to the living area. This may eliminate keyed entry during a certain the process, certain date or time and provide further verifications of individual or group access of the structure. A secured delivery area may be included in the structure so that packages delivery to the structure can be left at the structure in a secure location without the delivery worker having access to the living area.

Workers at the structure may be prompted to wear certain wearables that provide useful information to the system. For instance, individuals may be prompted to wear location tracking devices, such as GPS devices, Bluetooth, radio frequency identification (RFID) devices, ultra-high frequency (UHF) and/or beacon-based devices. The use of the wearables helps to perform geofencing within the structure. The location tracking provided by the wearable helps the system to monitor the location of individuals at the structure on an ongoing basis. The permissions may define what portions of the structure an individual may access. Ongoing monitoring may indicate that an individual is attempting to enter an area where the individual is not permitted. This may trigger a response as described herein. A signal may be sent to the vest or wearable to trigger a visual or audio cue that the individual is not in a permitted area.

In addition, individuals may be requested to wear wearables that track biometric information, such as heart rate, body temperature, respiration rate and blood pressure. This information may be tracked and stored on an ongoing basis. When the biometric data gathered from these wearables are outside an acceptable range, potentially indicating physical danger or injury, appropriate response actions may be taken, such as notifying the individual, notifying a supervisor, and/or contacting medical personnel. Collected data may be used to verify a multitude of factors such as reported accidents, incidents of theft, hours worked, and the like. In the event that an accident occurs, the system can record information about the accident such as the worker involved, the equipment being used, physical location, other worker(s) in the area, video capture from cameras in the area, materials involved, and/or tools and equipment involved and then it can record the information for reporting and future study.

The system may allow for the establishment of one or more geofenced zone that can be associated with delivery areas, worker entrance exit areas, task areas, storage areas, and any combination thereof. These areas could be monitored and established with access allowances or restrictions to control movement of material, individuals, and equipment to assist with the prevention of loss, mistakes, inefficiencies, and damage.

The system may control access to power by individuals at the structure. The system or structure may provide several power outlets or control power outlets to grant access to power as needed or warranted. Different levels of voltage may be provided as required and the system can determine, track and record power usage. The system can also restrict power usage during predetermined events including warranty confirmatory, safety situations, specifications, environmental conditions, and any combination thereof. Access to power can be controlled by the system or the power can include an intermediate power controller that can be controlled by the system. The power can be monitored for usage such as appliances, HVAC and the like.

The system, including a controller, may also interface with individuals to allow for the entry of notes and related details of a components, material, task, inspection, environmental condition worker, other task, process of individual or any combination thereof. For example, the system may allow a worker, occupant, owner, and inspector to capture images of notes, forms, documents, labels, and the like using various readers, sensors, and input devices.

Smart contracts may be provided that use the immutable ledger for each activity, transactions, or other event associated with the structure, occupant, worker, delivery, and owner and can execute upon satisfaction of terms of the event. For example, when a component is delivered from a supplier to the structure and received at the structure, a verification of the component with is virtual representation occurs and this event can trigger a smart contact that instates payment to the supplier. If an item is ordered by an individual associated with the structure, when the verification that the item was delivered, a smart contract can be initiated that will credit a suppliers account and debit the occupant's or other account. The supplier can include a supplier account that can received funds or other items of value from a payor account in exchange for providing goods or services that can be used or otherwise associated with the structure.

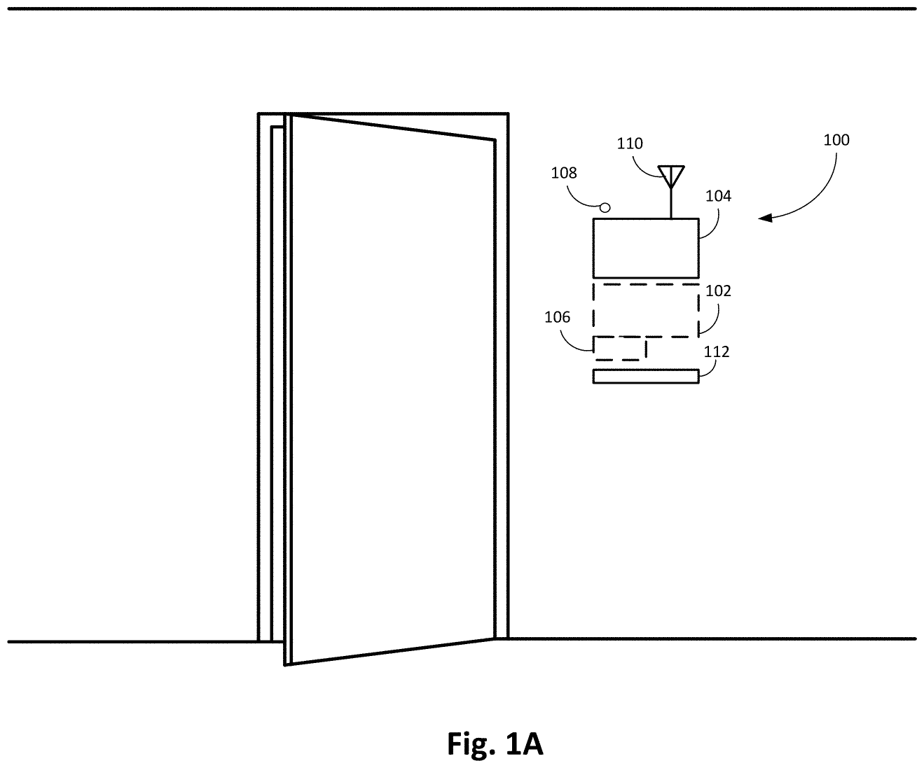

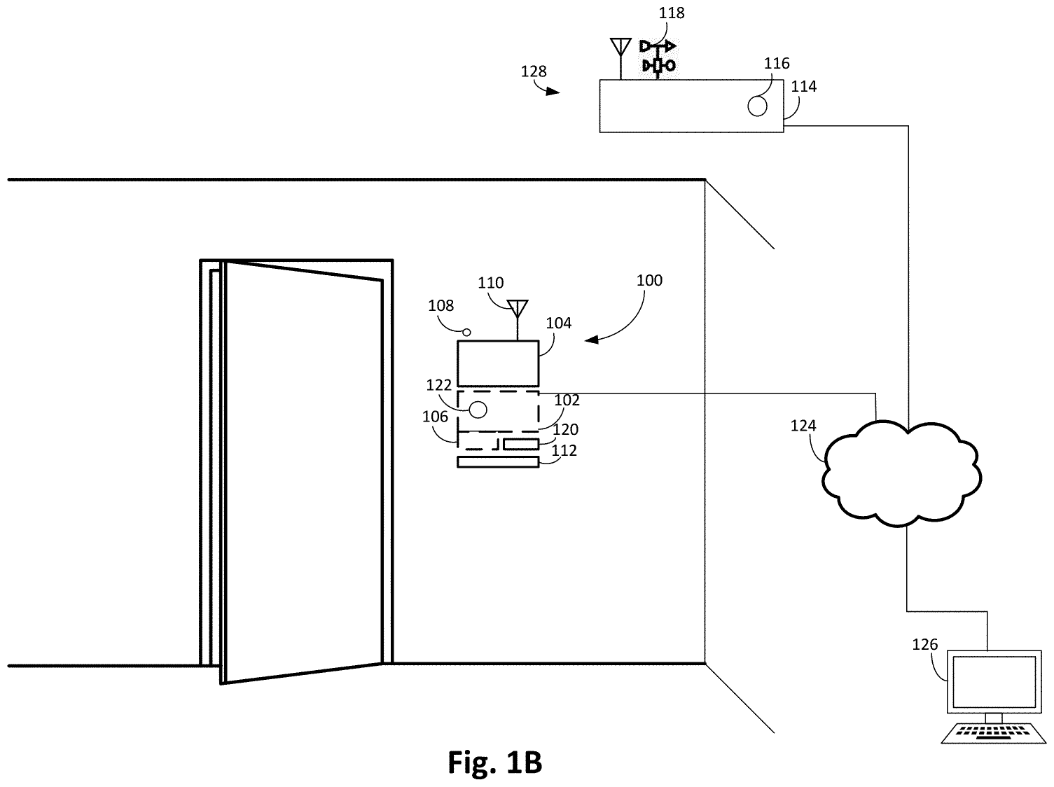

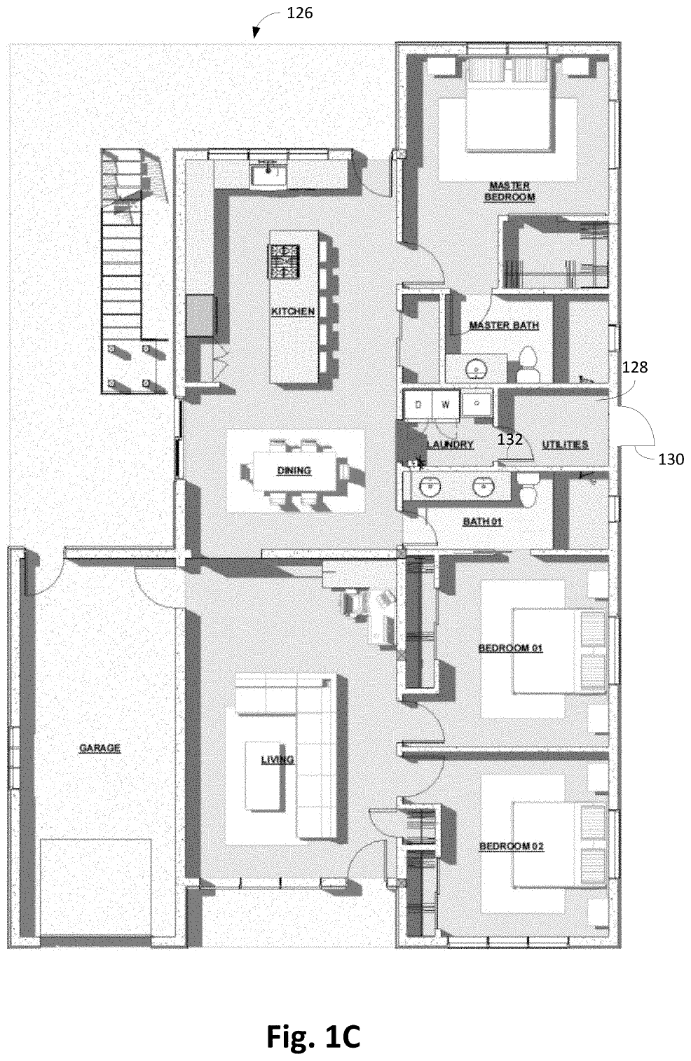

FIGS. 1A-1B illustrate an example of a system 100 can be integrated into a wall of the structure and can be uniquely associated with the structure in an exemplary embodiment. In system can include a controller 102 that can be attached to a wall or disposed within a wall and can be accessed through a panel. The controller can be in communications with a computer readable medium and remote computer system as well as other computer devices within the structure including appliances, HVAC, utilities, meters, and the like. The controller can be physically associated with the structure, virtually associated with the structure or both.

A display 104 and transmitter 106 such as a RFID can be associated with the structure by embedding it is a permanent fixture such as a concrete slab, foundation, structure, wall, partition, and the like. The system can read the information from the location marker and associate its actual location with the structure. The location marker can include an alpha, numeric or graphical information such as a number, letters, barcodes, QR code, physical or geographic coordinates (e.g., GPS coordinates), passive transmitter, active transmitter and the like. Each system can have a unique identifier and each structure can have a unique identifier.

FIG. 1A shows a first side of the system 100. The system 100 can include a camera 102 for obtaining images of materials, equipment, individuals or other items entering or leaving the structure as well as images of individuals along a perimeter. The camera 108 may capture biometric images upon which biometric recognition may be performed. Multiple cameras may be placed on or around the controller both interior and external to the structure. The cameras may have biometric recognition and motion detection capabilities. System 100 may include an addition to the camera 108 or instead of the camera 108, biometric-based identification devices that may be used to confirm the identity of individuals entering, leaving or on the perimeter of the structure. The system 100 may include an antenna 110 for communicating with a network including a wireless network, Wi-Fi network, Bluetooth, quantum networks, cellular network (e.g., 4G or 5G network) and any combination. The display may be a touchscreen display, upon which information may be displayed and entered. The display may include an integrated camera that may be used to capture images and that may be used in performing facial recognition of individuals. The display may also include or operatively associate with one or more integrated speakers for providing audio output, a microphone for receiving audio information to facilitate two-way communications to a remote location. The system 100 may include a scanner 110A for scanning items, such as deliveries, as will be explained in more detail below. The scanner 112 may be, for example, a QR scanner, an Optical Character Recognition (OCR) or a bar code scanner in some instances. The system 100 can be used for deliveries and inspections. A delivery person may scan delivered materials, components, packages, or other items via the scanner 112 and may interface with the system using the touch screen display 104. The system can include one or more displays, scanners, cameras, biometric sensors, sensors, and the like on the interior of the structure and the exterior of the structure. For example, a delivery worker can use the exterior scanner to scan a package delivery while an exterior camera can capture audio or video of the delivery worker at the time the package is scanned. The use of these two input devices included in an input assembly can provide improved verification that the package was delivered. The occupant can be notified that a package was delivered and placed outside the structure. The occupancy can then retrieve the package, scan the package using an interior scanner. An interior camera can capture video or images of the package and the occupant at the time the packer is scanned by the occupant. The exterior camera and the interior camera can be used to verify that the occupant retrieved the package inside without altering the package. When the package is opened, the interior sensors, including the cameras, can verify that the contents of the package is consistent with an order.

In one embodiment, multiple displays and sensors are positioned in and around the structure. The system can include an input assembly that can include sensors, biometric readers, cameras, transceivers, scanners, microphones, RF readers, Bluetooth, touch screen, microphone, data ports, and the like. An output can include a display, speaker, transceiver, printer, data ports, and the like.

In one embodiment, portions of the system can be disposed within a wall or other enclosures. An access panel may be provided to access these portions such as a breaker box, circuit board, fuses, and the like. The access panel may be under programmatic control in some instances to regulate access. If access is granted, the panel can be is unlocked, whereas if access is denied, the access panel can be locked. In some embodiments, access to the power supply may be controlled by controlling the flow of power to the power source under programmatic control from the controller. These control mechanisms may be used separately or in conjunction.

Referring to FIG. 1B the system can include an external assembly 114 that can include alarm indicator 116 that can be actuated as described herein. The housing can include a weather station 118 that can include an integrated or separate fluid (e.g., rain) collector. A biometric reader 120 can include an iris scanner, fingerprint scanner, palm print scanner, facial scanner or some combination. Display 104 can be proximity to or included in an input assembly such as buttons, scanners, microphones, cameras, microphone and the like. The input assembly and output assembly include a field receiver and indicators such as light. One or more cameras can provide up to a 360° field of view and include a wireless connection for transmitting images to the controller or a remote computer device. The images can also be used for input to the system including input allowing the system to identify delivered components, individuals, or other physical items. The system can include various sensors and cameras disposed around the structures for capturing images. One or more lights can include motion activation and photoelectric activation. Speakers 122 can be included to provide audio information to a user, worker, inspector, or other party using or near the system. The audio information can include instructions, alarms, and the like. The system can include a secondary power source, such as a battery, so that when power from the structure is not available, an alarm can sound, notification send to a remote computer device of other indication that the structure power source is unavailable. The system can include a hand scanner and a document scanner. The system can include a wired connection to remote computer devices or a transceiver to provide a wireless connection to remote computer devices.

The controller may be or may be in communications with many different computer forms including a desktop computing device, a laptop computing device, a mobile computing device, an embedded system, a smartphone, special computer device, custom computer device, or the like. A display may be integrated with the computing device or as a separate device, such as a liquid crystal display (LCD) device, a light emitting diode (LED) display device or other types of display devices on which computer information may be displayed. One or more biometric-based identification devices may be provided. As will be explained in more detail below, multiple biometric-based identification devices may be used. Network interfaces and a transceiver (e.g., modem) may be provided allowing the controller and various devices in the structures to communication with a wide area network 124, remote computer 126 and other devices, such as external assembly 128 associated with the structure. The controller may communicate with a local area network or a wide area network wherein the networks may be wired or wireless. A modem may be provided to communicate telephonically or over cable lines with remote computing devices.

The controller may be implemented in a distributed fashion and may include an alternative energy source. For example, solar panels, wind turbine(s), a battery or the like may be used. In one embodiment, the alternative energy source may be physically affixed to the structure or in communications with the controller. For example, solar panels or a cable to a wind power source could be configured to provide power to the system and/or can be affixed to the system or housing. Alternatively, a power line leading to the alternative energy source may be connected to the housing and system to provide power to the system, housing and associated components such as external power supplies.

The system may include a utility supply and control and a mechanism for turning the utilities, such as power, gas and/or water, on and off under a programmatic control. The system may include an internet data supply control and a mechanism for turning the access to this service on and off under a programmatic control. Programmatic control may be provided to grant or deny access to such resources or device in communications with the controller including appliances, HVAC, utilities, and the like. The controller may include or be in communications with a weather station 118 to measure current weather conditions, such as temperature, air movement, humidity, precipitation, barometric pressure, direct sunlight, and the like. Input from the weather station may be used to inform decision making by the system in some instances. Alternatively, the weather may be collected from third parties, such as from a weather service or other weather source. The sensor can be a wet bulb globe temperature adapted to measure, among other things, heat stress in direct sunlight, which accounts for temperature, humidity, air movement (direction and speed), sun angle and cloud cover (solar radiation).

Referring to FIG. 1C, an exemplary floor plan 126 of a structure is shown. The utility area 128 can be included in the structure and have access through an exterior utility door 130 and an interior utility door 132. The controller can allow for access through each door according to any number of factors including a maintenance request, work order, repair, construction, and the like. The utility area can include equipment such as an air handler, plumbing access, clean out access, solar power converters and exchanges, electrical junctions and the like. The floor of the utility area can be below the ground level of the structure so that in the event of a fluid leak, the fluid remains in the utility area without entering the rest of the structure. A drain can be included in the floor of the utility area to direct fluid to a proper disposal area (e.g., sewer). In one example, a maintenance request is created directed to a lack of hot water. The controller can receive information associated with the maintenance request including the worker identity who is to attend to the maintenance, arrival time or window, replacement part, and the like. The controller can retrieve biometric information of the worker and allow access to the external door if a sensor associated with the external door reads biometric information of the worker and matches it to the maintenance information. The controller can also only allow access during the arrival window for that worker. Sensors as described herein, can determine the workers identity, arrival time, maintenance or repair time, replacement part used, departure time and the like. Upon completion of the maintenance, a smart contract can be executed which can debit the occupant, owner or other account and credit the maintenance company account.

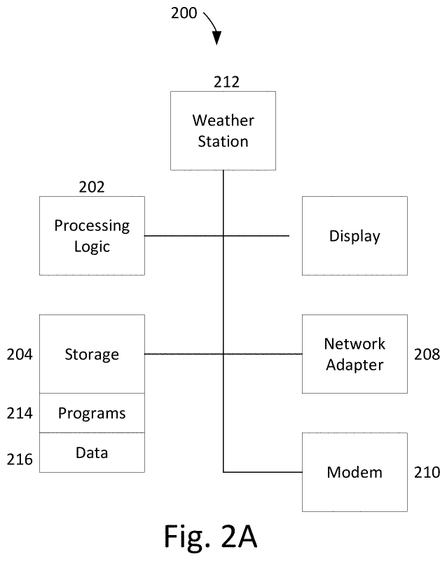

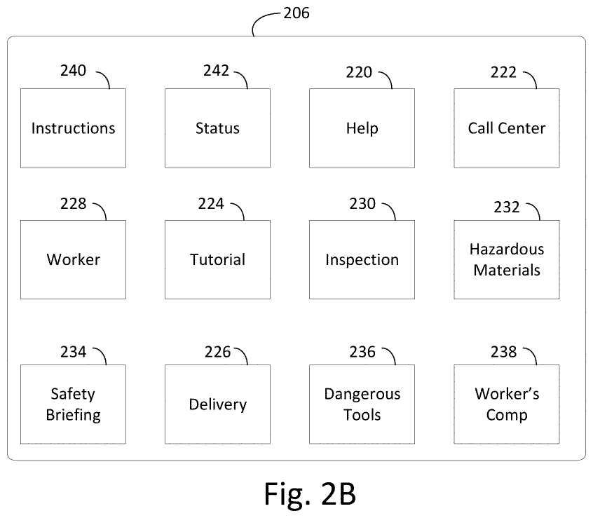

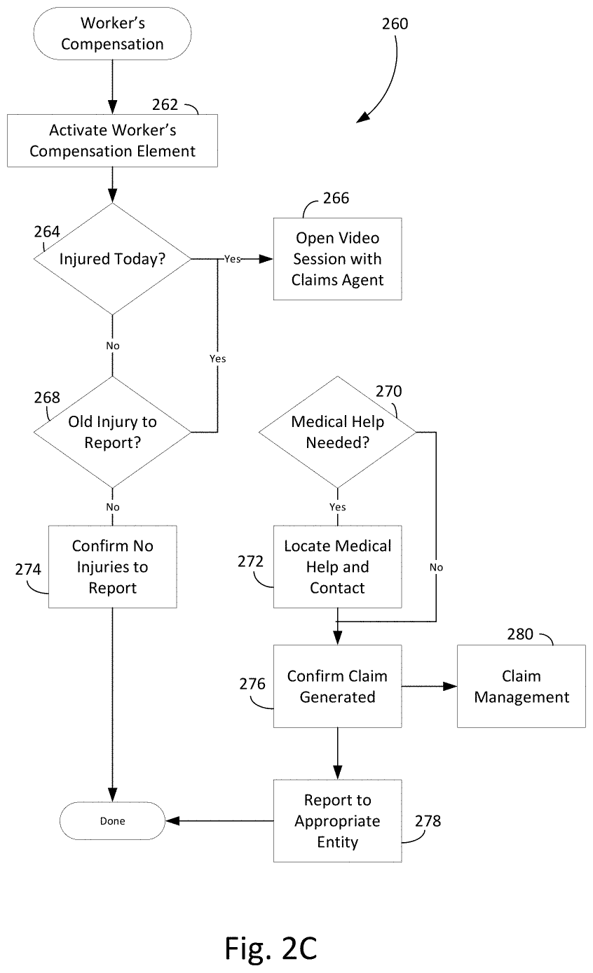

FIG. 2A shows an example of a controller 200 for the system. The controller may include processing logic 202, such as microprocessors, controllers, field programmable gate arrays (FPGA), application specific integrated circuits (ASICs) electronic circuitry, and other types of logic. The processing logic 202 performs the operations of the computing device 132. A storage device 204 may also be provided. The computer readable medium and/or data storage device 204 may take various forms, including magnetic storage, optical storage, etc. Storage capability 204 may include computer-readable media, including removable computer readable media, such as disks, thumb drives and the like, or disk drives, solid state memory, random access memory (RAM), read only memory (ROM) and other types of storage. The computing device may include a display 206, such as an LCD display, an LED display, or other types of display devices on which video information may be displayed. The computing device 200 may include a network adapter 208 for interfacing with networks and a transceiver and modem 210 for communicating wirelessly, over telephone lines or cable lines with remote devices. The processing logic 202 may use information stored in the storage device 204. In particular, the processing logic 202 may execute programs 214 stored in the storage and may access and store data 216 relative to the storage device 204. The computational functionality of the system described herein may be realized by the processing logic 202 executing the programs 214.

FIG. 2B shows an example of a user interface on display 206, such as found in or in communications with the controller. The user interface may include activatable elements. A user may depress these activatable elements or select these activatable elements using an input device, such as a mouse, keyboard, touchscreen, or the like, to activate the components. The display 206 may include a help element 220 that may be activated to obtain help information regarding use of the controller. It may also contain real time project or process plans. It may also include “how to” assistance including videos related to the various projects, stages, processes, and tasks performed at the structure. The user interface on the display 206 may also include a call center activatable element 222. Selection of the call center activatable element 222 may cause a call to be initiated with a call center so that the individual using the system 100 may have a telephone and or video conference with personnel at the call center. The user interface on display 206 may also include a tutorial activatable element 224. Selection of the tutorial activatable element 224 causes a tutorial to be displayed to teach the individual about operation of the housing.

A list of hazardous materials at the structure may be activated by activating element 232. This list is updated as hazardous materials are delivered, removed, or consumed. Access to hazardous materials may also be controlled via the controller. The display may show instructions 240 for completing certain tasks or other information. A status of tasks and materials can be displayed at 242. For example, a worker can view the display and receive status information about materials such as anticipated delivery, route information, or tasks as well as the status of tasks including performance steps, start times, competition times and the like. A hazardous material record can be created and recorded on the immutable ledger that includes a verifiable pairing to the hazardous material.

The user interface on display 206 (FIG. 2B) may also include a safety briefing activatable element 234. Activation of this element 234 results in a safety briefing being displayed on the display 206. The user interface on display 206 may include a dangerous tool activatable element 236. Activation of this element 236 causes the display of a list of dangerous tools at the structure. An individual must have the proper authorization or certification to use such dangerous tools. The authorization or certification may be stored with the permissions.

The controller may include software which allows a tool to be coded or assigned to authorized worker. Each tool can have a verifiably paired virtual representation associating the specific tool with the virtual representation. This can be verified by the system through recognition of the tool from a reader or sensor. The tool supplier record can be created by the tool supplier and include a virtual representation associated with the tool and store the virtual representation paired with the tool on the immutable ledger. When the tool arrives at the structures, the system can retrieve the tool supplier record and determine of the tool requested if the actual tool that arrived. A tool verification record can be created and stored on the immutable ledger representing that the tool delivered and received matches the tool supplier record.

The controller can also allow or prevent access to the structure. When a worker arrives at the structures, the selection of the worker's compensation activation element 238 may be required at the beginning and end of each workday or when an injury occurs to track the worker. All workers may be required to enter this information each day. If the worker indicates that the worker has been injured 264, a video session with a claim agent can be initiated at 266. The claim agent may gather information to initiate any claim processing. The claim agent may determine if medical assistance is warranted 270. If medical help is warranted, the location of appropriate medical help can be identified based on a location of the structure (e.g., based on proximity and type of injury) and contact is made with the medical assistance (e.g., calling of an ambulance, hospital, or urgent care facility) 272. The facilities may be chosen to be “in network” for the worker's compensation carrier. A confirmation of the claims may be generated 276 and sent to claims management 280. In addition, a report may be sent to the appropriate entity or authority at 278. The steps 266, 270 and 272 may also be performed in the instance in which the individual has an older injury to report 268. Where there is no injury to the individual, the lack of injury is reported 274.

Shipping or delivery company personnel may activate the delivery activatable element 226 (FIG. 2B). This causes a delivery functionality to be displayed where delivery notes may be added and where information may be gathered from the delivery person regarding a particular delivery. A worker activatable element 228 may be selected by workers. Selection of this activatable element 228 causes the activation of the worker functionality whereby the worker may sign in, request tools, equipment, power or materials, leave notes or the like.

An inspector activatable element 230, may be activated to cause the inspector functionality to be activated. The inspector functionality may enable an inspector to add inspection notes, provide electronic inspection certificates and the like. The system can provide reports that can be automatically generated from the existing data described herein as well as notes manually added during the construction process. The reports can be generated at predetermined times such as daily or upon completion of specific tasks.

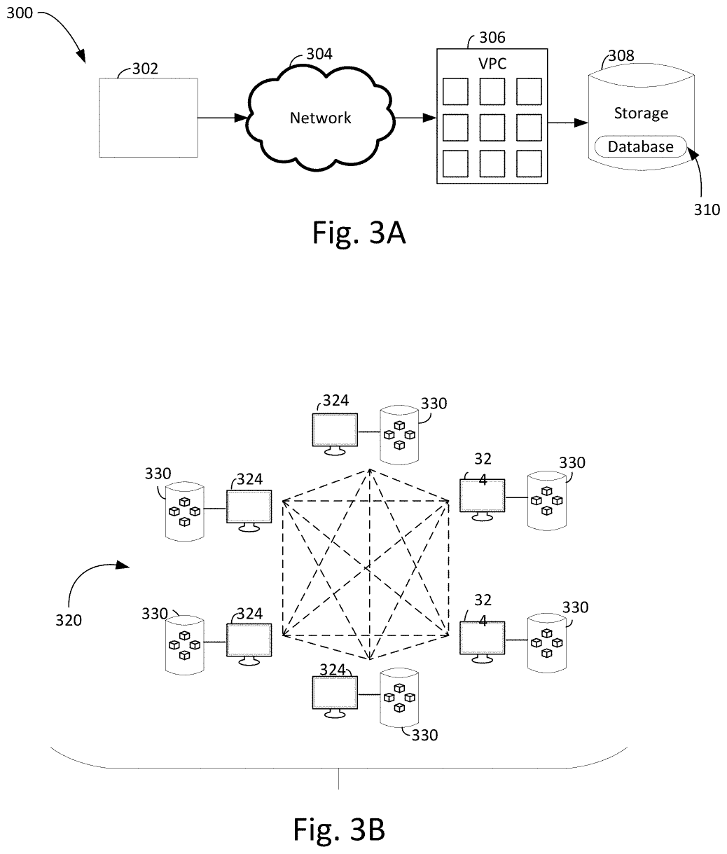

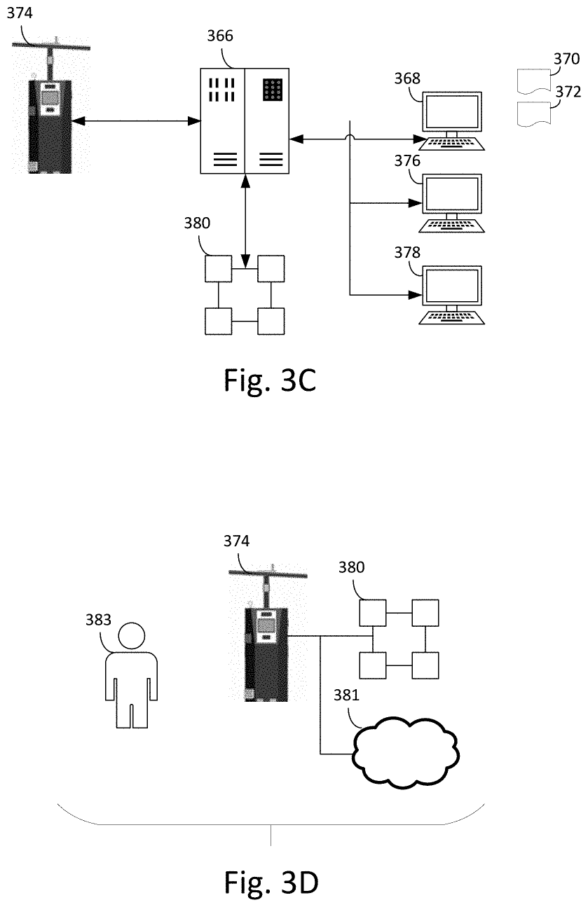

As shown in FIG. 3A, the exemplary embodiments may be implemented in a decentralized computing environment 300, that may include distributed systems and cloud computing. FIG. 3A shows one or more systems 302 that may be in communication with a remote cluster 306 via a network 304. The cluster 306 may store information received from the system 302 and provide added computational functionality. The network may be a wired network or a wireless network or a combination thereof. The network 304 may be a secure internet connection extending between the system 302 and the cluster 306, such as a virtual private cloud (VPC). The server may be a computing device and can be in communications with the site computer device. The cluster 306 may include access to storage 308. The storage 308 may include a database 310 in which information regarding a structure is stored in a consistent manner.

FIG. 3B shows diagram 320 of an example of a peer-based network where an immutable ledger 330 is broadcast and shared among the nodes 324. This network may be resident in the VPC cluster 306 (FIG. 3A) or in the network 304 for example. The nodes 334 may represent computing resources, such as server computer systems or other computing systems. Each node that has access to a copy of the immutable ledger 330.

Referring to the server 366 can be in communications with design computing device 368 that can be used to transmit design information 370 and material requirement record 372 to the server and housing or structure computer device 374. The information from an owner, occupant, worker, designer, supply company or other third party can also be stored on the immutable ledger and retrieved by the system. The controller can be configured for receiving a maintenance request or delivery request from the third party of immutable ledger representing physical articles to be delivered to the structure, creating an actual material requirement record for the structure representing the actual material delivered to the structure by a supply company, creating a final material record according to a difference between the material requirement record and the actual material record, receiving material installation information from the supply company representing the installation criteria for the actual materials delivered to the structure, receiving installation verification information representing the actual material was installed by an authorized installer 383 and under a set of compliant environmental conditions, including weather conditions, receiving warranty criteria from the supply company representing the warranty requirements associated with the actual materials installed, determining if the warrant criteria are met.

Conditions associated with a project, structure, location marker, delivery, pick-up, worker, process, or task can include physical location (e.g., GPS coordinates), weather conditions, impacted workers, impacted materials, impacted equipment, date and time, duration, pre- and post-events (e.g., chronologically relevant action(s)), managers and supervisors on site and/or responsible. An insurance event can be an event, act or omission that affects the risk associated with insurance coverage. An insurance event can include an injury, loss, potential for an injury or loss, failure to supervise, misreporting of materials, workers, and the like that could cause an insurer to pay a claim or create the potential for an insurer to pay a claim.

Processes, projects, and task specifications, which may be needed for compliance with warranty, insurance, design, specifications, inspection, and other requirements, can be received at 376 and regulatory code can be received from a regulatory computer device 378 either directly or from the immutable ledger. The regulatory requirements can include approved materials that are approved by regulatory entities, such as governments, or designers, such as architects. Regulatory requirements can include product safety codes, building codes, fire codes, labor standards, building permit requirements, building and labor licenses, and the like. The regulatory requirements can include processes and procedures for handling, use, installation and assembly. For example, during building construction, opening for windows and doors on second floors or higher should have safety railing installed.

The various computer devices, including the server and controller (e.g., system, controller, and any combination), can be in communications with immutable ledger 380. The immutable ledger can include a distributed ledger, immutable database, block-chain structure, and the like. The communications between the various computer device, including the server and the site computer device and immutable ledger can be a global communications network, wide area network, or local area network, delivered to a computer readable medium from one device to another (e.g., USB drive, CD, DVD) and can be wired or wireless.

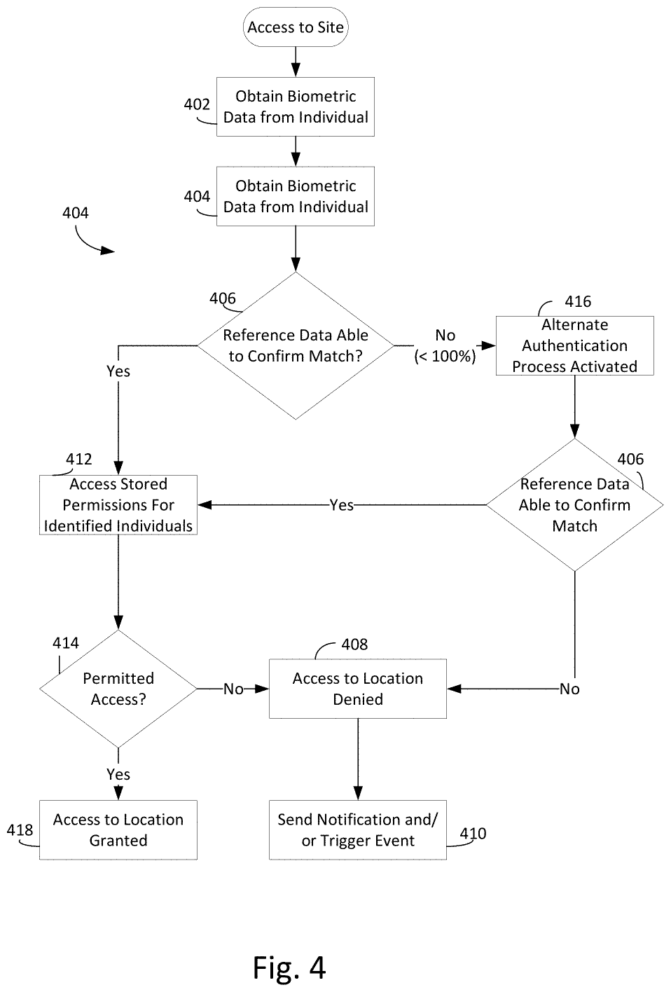

FIG. 4 shows a flowchart 400 identifying steps that may be performed in exemplary embodiments regarding this functionality of the system. Initially, biometric data is obtained from an occupant, owner, worker or other individual that is seeking access to the structure 402. In some exemplary embodiments, a camera 102 may capture an image of an individual and facial recognition may be performed. The biometric data in this case is the facial image of the individual. In other exemplary embodiments, the biometric data may be, for example, fingerprint data, hand scan data, voice print data, retinal scan data or the like, gathered by appropriate biometric-based identification devices. The obtained biometric data is stored, and then previously stored data is accessed from storage to compare biometric data for known individuals and to attempt to identify the individual 404. A comparison may be made between the gathered biometric data and the known biometric data to determine if there is sufficient closeness for there to be a match. Information regarding the identity of the individuals for which the biometric data is stored is also stored in the storage device. A determination is then made whether there is a match or not 406.

If there is not a match 406, a manual process may be executed, or an alternative authentication process may be deployed 416. If this alternative authentication fails to produce a match 406, access to the structure may be denied 408. In addition, a notification may be sent to a responsible party and/or an event may be triggered, such as contacting security or law enforcement officials 410. If the alternative authentication process produces a match, the process proceeds to 412.

The system may store permissions for each individual accessing the structure. These permissions may identify the dates and times where the individual is given access to the structure. In addition, the permissions may specify what tools, equipment, or materials the individual can access. The permissions may specify whether the individual can use a power supply and may specify what portions of the structure the individual is permitted to access. These permissions may be accessed to determine the permissions for the identified individual 412. If the permissions indicate that access is permitted 414, the individual may be granted access to the structure 418.

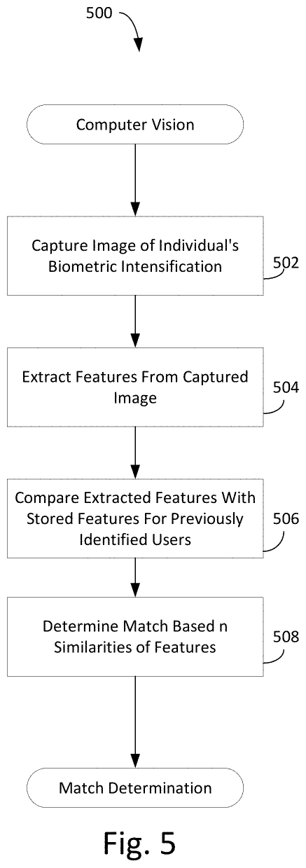

FIG. 5 shows steps that are performed in a case of computer vision for 402, 404 and 406 of FIG. 4. The flowchart 500 begins with 502 in which an image of an individual is captured for biometric recognition. This may be captured by a number of different types of image capture devices, including an intermittent video camera, still camera, iris scanner, facial scanner, fingerprint scanner, or other type of capture device. In the case where an image of the face of an individual is captured, identifying features may be extracted from the captured image 504. In other words, unique facial features that help to identify an individual are extracted from the image. The image may be filtered and/or normalized. The features are then compared with the stored features for identified individuals 506, determination is made whether there is enough similarity for there to be a match.

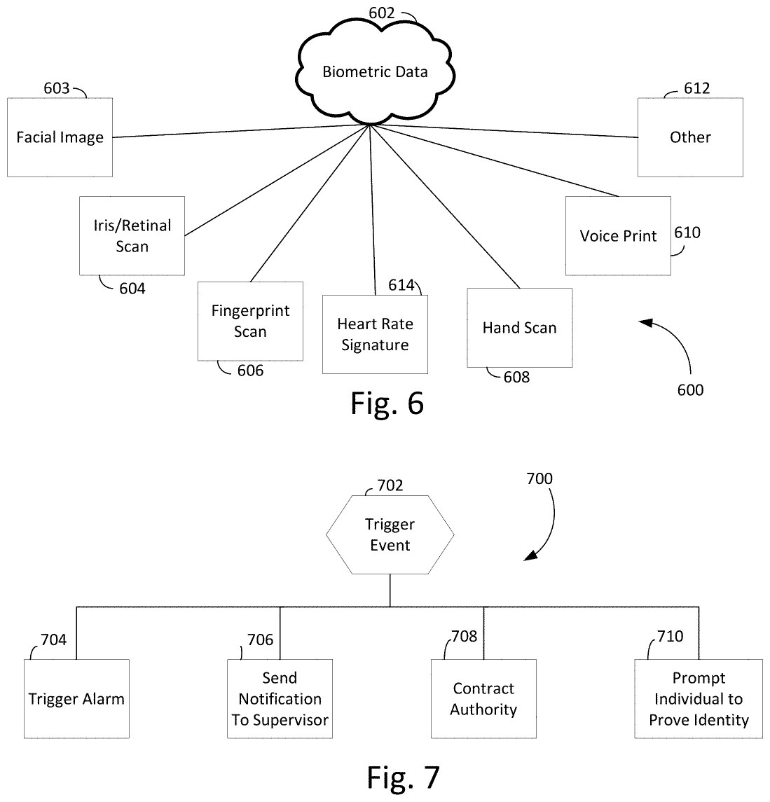

FIG. 6 shows a diagram 600 that illustrates various types of biometric data 602 that may be obtained by biometric-based identification devices at the structure to attempt to identify individuals. Biometric data may include facial recognition 603, an iris/retinal scan 604, a fingerprint scan 608, a hand scan 608, a voice print 610 or heart rate signature 614. It should be noted that other types 612 of biometric data may also be used in exemplary embodiments to help identify individuals uniquely. Also, an individual may be required to provide multiple types of biometric data in some instances.

When individuals attempt to access the structure and are not granted access, certain events may be triggered (see 410 in FIG. 4). FIG. 7 shows a diagram 700 that provides an example of different types of triggered events 702. One type of triggered event is an alarm 704. This alarm may include visual alerts, audio alerts and any combination thereof. The alarm may be a silent alarm to individuals. Another event that may be triggered is to send notifications to a supervisor for the structure 706. The supervisor may, for example, receive an email, a text, a phone call or another notification that someone is trying to access the site that is not permitted. A triggered event 702 may also include the contacting of law enforcement or a member of a security service indicating that an unauthorized party has tried to access the structure. Lastly, a triggered event 702 may include prompting the individual to produce proper identifying information to an official at the site or to a scanning device at the housing 100.

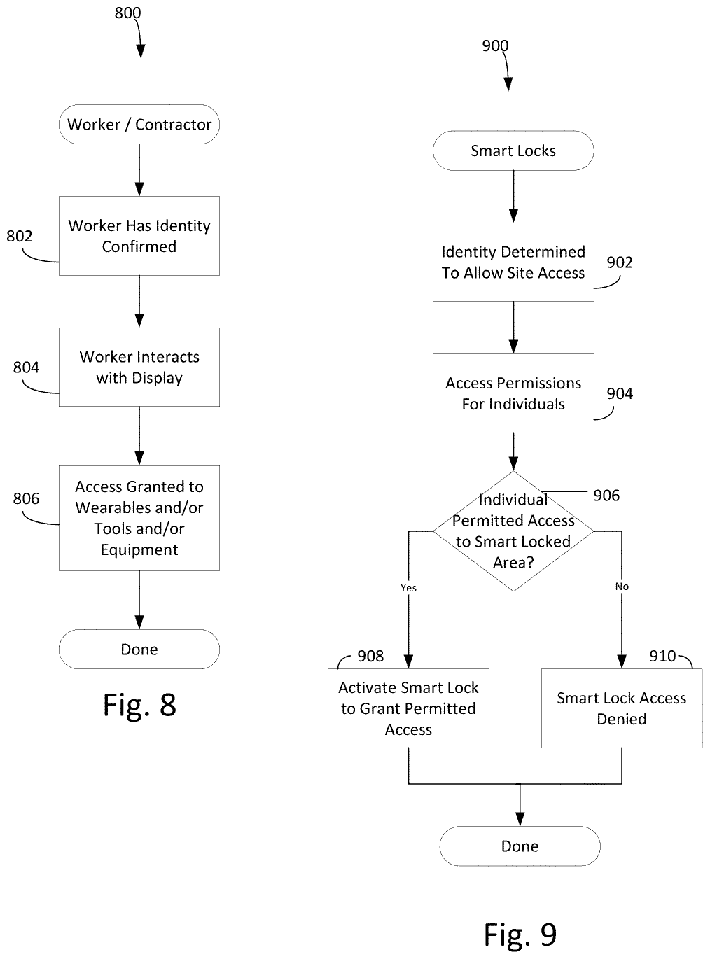

FIG. 8 shows a flowchart of the steps that may be performed to ensure that a worker gains access to the appropriate items once they have been granted access to the structure. As shown in the flowchart 800 of FIG. 8, initially the worker has their identity confirmed, as has been discussed above 802. The system may offer an alternative touchscreen option to place a call to the appropriate party should the software fail to verify and otherwise authorized the worker. The worker may be prompted to interact with the display, such as the touchscreen 106B (FIG. 1) to register and to indicate whether they seek certain items. For example, with the user interface of FIG. 2B, the worker may activate the worker activatable element 228. Access is then granted to wearables and/or tools and/or equipment 806. The wearables, the tools and/or equipment may be stored in sheds or in other secured locations under the control of smart locks that may be controlled by the computing system of the system.

FIG. 9 provides a flowchart 900 of the steps that may be performed relative to smart locks at the structure. The individual, such as a worker, has his/her identity determined and has access to the structure 902. The system may offer an alternative touchscreen option to place a call to the appropriate party should the software fail to verify an otherwise authorized individual. The permissions stored for the individual are accessed 904. A determination is made whether the individual is granted access to a smart locked area 906. If the individual has permission to access the area 908, the smart locks may be de-activated to unlock the area, such as where wearables, tools or equipment are located. Before the individual can use the tools/equipment, the individual may first be required to wear some of the wearables and to scan the tools or equipment to indicate that they will be using the tools or equipment. If the individual lacks the proper permissions to access the area, then access to the area is denied 910, such as by keeping the smart locks locked. In one embodiment, the tools and equipment used can be determined so that the proper tools are used for installation of materials if required for warranty requirement compliance.

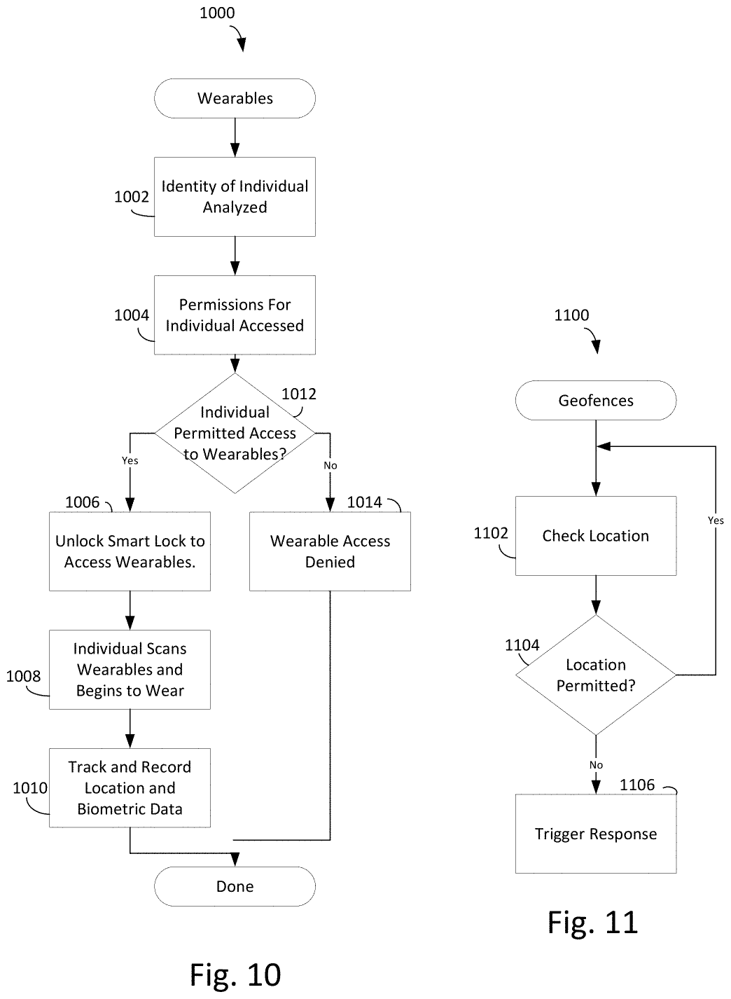

FIG. 10 contains a flowchart 1000 illustrating the steps that may be performed to obtain the wearables. For some individuals, a wearable can be provided to the individual prior to interaction with the system and used to register with the system. This individual can receive the wearable and upon approaching a housing, be registered with the system when the individual reaches a certain proximity to the system.

The wearables may include safety gear, such as hard hats, gloves, goggles, vests, and the like, as well as wearables for tracking and obtaining biometric data. The identity of the individual is confirmed 1002, and the permissions for the individual are accessed 1004 and a determination is made based on the permissions if the individual should be granted access to the wearables 1012. The system may offer an alternative touchscreen option to place a call to the appropriate party should the software fail to verify an otherwise authorized individual. If it is determined that the individual should be granted access, the smart lock for the storage location of the wearables is unlocked 1006. The individual may then be prompted to scan information regarding the wearables so as to register the wearables and associate the wearables with the individual 1008. In one embodiment, the system may use one or more images of an individual to determine if the individual has the necessary safety equipment. For example, an image of the individual taken by the housing can be used to determine if the individual is wearing a hardhat and generate a warning if no hardhat is present can be generated and transmitted appropriately.

The system may require that an individual wear certain safety equipment in the form of wearables. The system may record what wearable the individual scans. The system tracks and records the location and biometric data gathered by the wearables 1010. The wearables may include smart vests, bracelets, badges, sensors, and the like that provide location information and biometric data, such as heart rate, body temperature, blood pressure, breathing rate, gyroscopic informatic and/or other information. These wearables assist the system in tracking the location of individuals of the structure. These wearables also help to track the biometric data of individuals. The biometric data may be helpful in identifying that an individual is experiencing an accident, a health event or is idle. The biometric data is stored so that a record of the biometric data can be kept. If in 1005, it is determined based on the permissions that the individual should not be granted access, then access to the wearables is denied 1014.

The use of the wearable to track location helps to facilitate use and installation of material to determine warranty requirement compliance. The wearable information can be combined with geofencing in an exemplary embodiment. The geofencing enables the system to track and limit access to locations of individuals at the structure. The structure may be partitioned into areas where different permission rights are given for the various areas. For example, a mechanic can be given access to the repair section of a structure and denied access to the paint area. A worker can be given access to the point-of-sale portion of a structure and denied access to the loading docks.

As shown in FIG. 11, a flowchart 1100 shows some of the steps performed automatically and can be performed without notification to an individual. The process begins with the checking of the location 1102 of an individual. A determination is made in 1104 whether the individual is permitted to be at that location. If the individual is not permitted to be at that location, a response is triggered 1106.

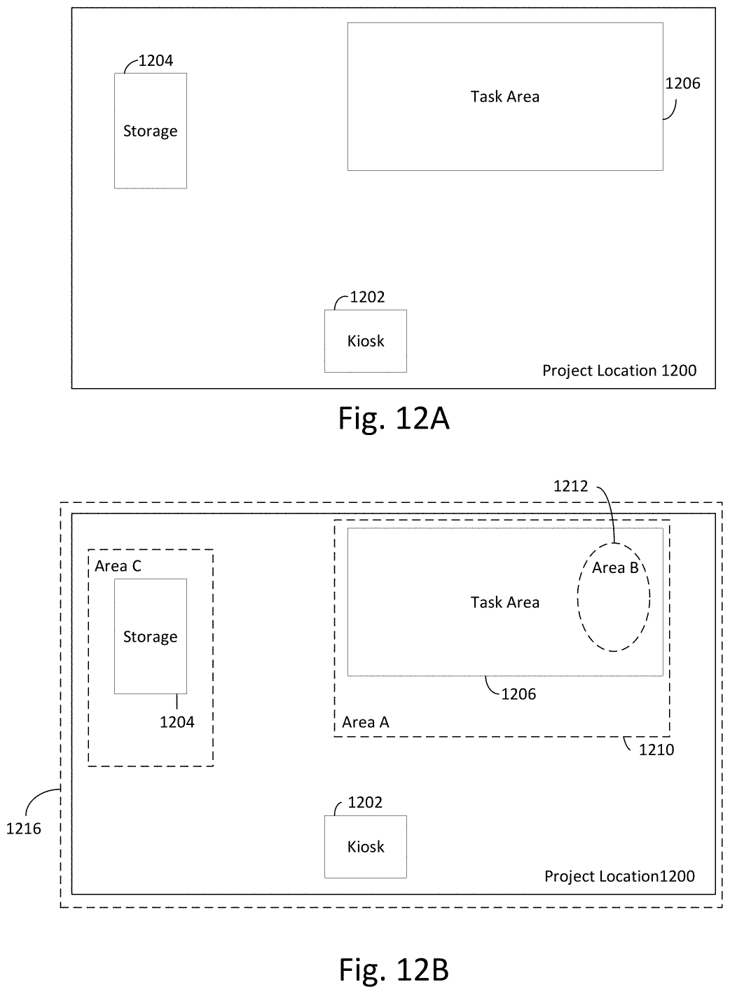

To help illustrate an example of geofencing, FIG. 12A shows an illustrative structure 1200. The structure 1200 may include a housing 1202 for the system as well as storage location 1204 that can be a building, trailer, shed or the like. The storage location 1204 may hold tools, equipment, wearables and/or materials. The structure 1200 may also include a task location 1206. The task location may be where tasks are performed using materials to produce a good or offer a service.

FIG. 12B shows an example of different areas that may be established for geofencing at the structure 1200. Area A shown a boundary 1210 may include the entirety of the project that is under construction 1206. Area B 1212 may be a portion of the project, such as the kitchen. Area C 1214 may be the shed and area D 1216 may be the entire structure. Individuals may have access to none of these areas or to a subset of these areas, including all areas.

When a worker enters an unauthorized area, an audio or video response can occur on the wearable. For example, a smart vest may include lights that may flash or may be continuously illuminated in response to a party being outside the permitted areas on the structure. Another option is for an audio alarm or a video alarm to be triggered at the structure rather than on a wearable. A further option is to send a notification, such as a phone call, a text message, an email message, or other notification to a supervisor on or off the structure. Yet another option is to take disabling measures relative to the individual. The disabling measures could entail triggering locks or disabling equipment by shutting off power or the like. The geofencing can also be used to determine where the material will be delivered, stored, and installed. The geofencing, along with date and time information, can assist with the determination whether the material was handled or installed by a licensed, experienced, approved, authorized, or otherwise preferred worker.

Initially, the identity of the delivery person is confirmed to indicate that the delivery person is the appropriate party and is permitted access to the structure 1302. For example, a serial number or other identification indicator may be scanned or read off the delivered items. In addition, information may be entered by the delivery person using the housing, such as by entering information via screen (FIG. 1A) 1304. The location of delivery, the date of delivery, the time of the delivery, the quantity of the delivery, the identity of the delivery person and the weather may be recorded as part of the information that is kept regarding the delivery. This information can be used to track and confirm deliveries as well as to understand the conditions when the delivery was made.

The deliveries may utilize various scanning and reader technology. In FIG. 1A, a scanner may be provided. Diagram 1400 illustrates different types of inputs 1402 that may be used for assisting gathering information regarding deliveries. A machine vision system 1404 may be provided. The machine vision system 1404 may capture an image of the delivered items and process the image to determine the nature of the items that were delivered as well as the quantity of items. Moreover, the machine vision system may capture an archival image that may be indicative of the state of the items when they were delivered. A QR code scanner 1406 may be used where QR codes are on a delivered items or documentation. Similarly, a bar code scanner 1408 may be used where bar codes are on the items or on documentation delivered with the items. Still further, an RFID reader 1410 may be provided to gather information regarding the delivered items.

The delivery person may interface with housing via display 1300 to provide delivery information. Flowchart 1500 illustrates some of the steps that may be performed in such an instance. Initially, the delivery person may access the housing 1502. The delivery person may enter a note(s) regarding the delivery, such as what was delivered and the state of the items that were delivered 1504. This information may be entered, such as through the display 106A (FIG. 1A) which can be a touchscreen. The delivered items are imaged, scanned or read 1506. As was mentioned above, a number of different types of input technology may be used on the delivered items. Therefore, delivery information is then stored in records that may be accessed subsequently 1508.

The inspector may interface with the system. Initially, the identity of the inspector may be confirmed using the biometric data 1602 or manually using the touchscreen on the system. The inspector then performs the inspection of the appropriate portion of the structure 1604. The inspector then accesses the system, such as through the system at 1606. The inspector then may record notes and/or post certificates or notices at the system 1608. Additionally, the inspector may use technology available via the system such as OCR scanner or the like to capture appropriate information the inspector may have written during fulfillment of the reason for being on the site.

The system may include a still camera(s) or a video camera(s) that can be included in the system. A software interface to the camera may be provided to enable authorized external parties to gain access to the camera 1702. A party requests access to the camera via the interface over the network 1704. For example, a bank official may wish to view the structure before authorizing release of funds or before granting a loan. A determination is made whether the party is permitted access by accessing permissions 1706. The system gathers a great deal of information over the course of time. At least a portion of this information is persistently stored to compile a record of activities at the structure. This record can be useful to prove activities after the fact. The activities that are recorded may drive workflow and scheduling at the structure to improve efficiency. If the party is permitted access, access is given to the party so that they may receive a captured image or video data 1708. Otherwise, access to the camera by the party is denied 1710.

Systems at adjacent locations may be used in conjunction with each other so that image capture devices, such as still cameras or video cameras, from multiple adjacently situated systems can be used in conjunction in another system. Video feeds or still images may be obtained from the image capture devices from multiple systems. The video feeds or images may then be processed, such as by the cluster described above, using software such as motion detection software, thermal image analysis or other image analysis software to identify activity that may warrant a response. For example, the analysis may identify a large living object moving near the periphery of multiple structures. The system may then, based on the analysis, determine if action needs to be taken. If an action is needed, then the action is taken. If not, the capture of the images or videos may continue. Examples of actions include sounding an alarm, contacting law enforcement, contacting a security team or the like.

The environmental conditions, including weather conditions, may trigger different protocols that are performed by the system. For example, inclement weather, very cold weather or very hot weather may trigger protocols to ensure worker safety and productivity as well as the protection of structures, materials, and equipment at the structure. Initially, the system receives environmental data 1802. A check is made whether the weather data values trigger any protocols 1804. If a protocol is triggered, notices and alerts may be sent as part of the protocol to the appropriate parties 1806. For example, if the temperature is over 90° F., a worker performing maintenance exterior of the structure may be sent a notice to take a break every hour and to hydrate. Such notices can be generated according to the component specifications in the history of audit trail, when components are confirming or nonconforming, environmental conditions and other events. For example, if it is too cold, workers may be prompted to wear gloves and hats and to spend time in a heated space every hour. In still another example, if severe weather is approaching, workers may be prompted to secure the structure and seek a safe location. In a final example, workers may be prompted not to take actions, such as pouring concrete, applying paint, or applying stucco, in certain weather conditions.

As has been mentioned above, a great deal of information may be collected and stored during the project, process of task for reference during or after the project, process or task is completed. The information obtained during activity, transaction or other events associated with the structure may be stored on or referenced from immutable ledger. The information may be stored on an ongoing fashion, in databases as described below, and may be referenced in an immutable persistent fashion on the storage. This information may help resolve disputes between parties involved on the project or process. For example, suppose the occupant determines that the wrong materials were delivered. Since there is a complete record references on the immutable ledger of all deliveries, these records may be accessed to resolve the dispute. Insurance providers may access injury records referenced on the immutable ledger to settle or confirm claims. Disputes regarding pay among workers may be resolved by checking the recorded hours on site to determine the appropriate pay for the workers. Inspection records may be accessed to confirm that proper inspections were carried out and passed.

The advantages of this system can be shown by way of example. Suppose that an oven in the structure fails three months after an occupant occupies the premises. Since the immutable ledger has information concerning the manufacturer, design, supply, delivery, installation and inspection of the oven, complete warranty information is available, the owner, supplier or repair entity can reference the immutable ledger to have the oven repaired or replaced. In another example, suppose that gutters on the home leak six months after an owner occupies the premises. The owner can reference the immutable ledger, assembler or manufacturer warranty and repair or replace the gutters. The assembler might also have a remedy against the installation worker, based on evidence that materials were not installed properly, the manufacturer guidelines were not followed, conditions precedent were not met or other errors or omissions. Further, the shipping delivery record may indicate that the materials delivered were different from those specified in the material requirements record.

The information referenced in the immutable ledger may also be accessed from a computing device of an owner, end user, customer, integrated and the like. The computing device may be, for example, part of a home maintenance system that manages and controls home systems, such as heating, air conditioning, lighting, an alarm system, or the like. The computing device may be part of a smart home controller and may interface with appliances and other items that are interconnected via a home control network. The computing device may include a document management system for securely storing the transferred information. The computing device may be a facilities management system, or operations system associated with the structure.

In exemplary embodiments, information may be gathered from and sent to multiple parties including a managing company responsible for the management and oversight of the project or process, a design company responsible for developing a design for the home and developing the material requirement record for the project or process, a supply company which analyzes the design and materials for the project or process, and the individuals or entities responsible for performing and completing the project or process.

Component information and other information associated with the structure can include component warranty information for components used in the building of the structure. The supply company database may also store component information. This may include bill of lading histories and barcode, RFID values, UHF values and/or QR code scan histories. The material list (such as a bills of lading) for the structure and the barcode/QR scan codes for delivered items for the projects may be hashed and the resulting hash value(s) passed to the management company database for reference on the immutable ledger. Further, confirmation of what was specified by designers was delivered to the structure and installed according to the manufacturer's specifications so that a project or process can be placed under warranty.

Occupant, owner and worker information may include information regarding licenses for workers, hire dates for workers, employment end dates for workers and other information, such as names, photos, etc. The worker information can include information that the worker is in compliance with applicable laws (including federal and state), in compliance with contractual obligations, properly licensed, of legal status, of sufficient experience, within application restrictions such as a limit on hours worked during a 24-hour period, authorized for the structure and any combination. The worker or laborer information may be sortable by keys such as TMS # to identify workers for a project.

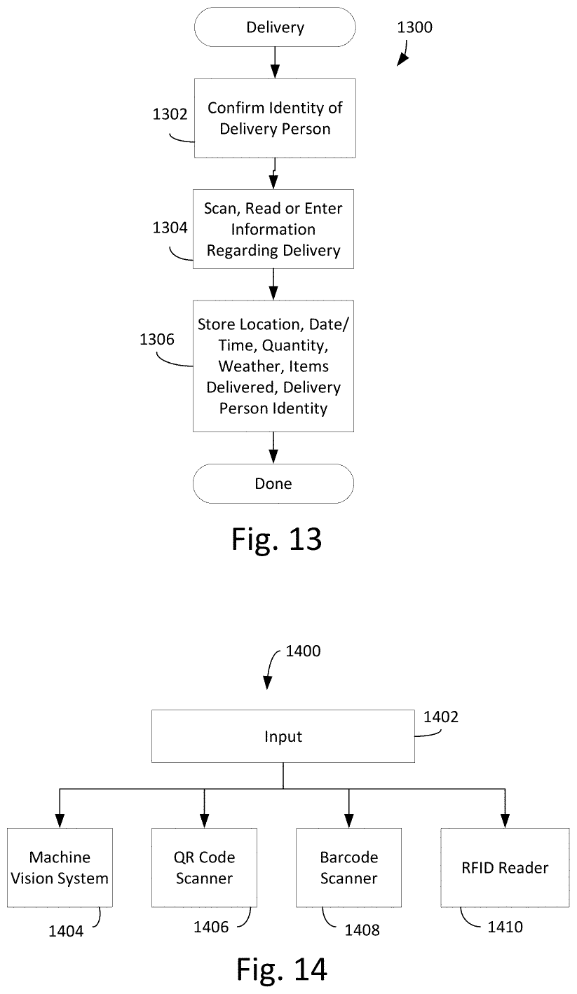

Information referenced on the immutable ledger may originate from third parties. FIG. 13 shows a diagram 1900 of additional sources of information from other stakeholders. For example, information may originate from a lender database 1906. For example, hashed versions of a deed for a building 1914 and the mortgage 1916 on the structure property may be stored in the lender database 1906, transferred to the management company database 1904 and referenced on the immutable ledger 1902.

Information may originate from an insurance company database 1908. A hash of an insurance policy for the project 1918, a hash of any insurance policy changes for the project 1920, a hash of policy start date 1922, a hash of policy cancellation date 1924 and a hash of policy change dates 1926 may be stored in the insurance company database 1908. The resulting hash values 1918, 1920, 1922, 1924 and 1926 may be transferred to the management company database 2804 and referenced on the immutable ledger 1902.

Information may also originate from a property management database 1910. Hashes of a current rental agreement 1928, rental agreement particulars 1930 (such as agreement number, dates and amount paid) and notice(s) of missed payments 1932 may be stored therein. Information can include utility consumption and expenses such as utilities, insurance, taxes, maintenance, and the like.

The hash values 1928, 1930 and 1932 may be added to the management company database 1904 and ultimately referenced on the immutable ledger 1902.

The supply company 2010 is responsible for the intake of materials specified in the material requirement record that are needed for the project or process. FIG. 14 shows a diagram 2000 of steps taken in the material intake process 2002. Materials arrive 2014 at a processing area of the supply company 2010 from the manufacturers and/or distributors. Items such as barcode stickers, QR code stickers, Bluetooth beacons, UHF stickers and/or RFID stickers are applied to the materials 2006 so that the materials may be identified and tracked. The materials with the stickers applied are placed in a designated area for packing 2008. The materials may then be packaged for shipment to the structure.

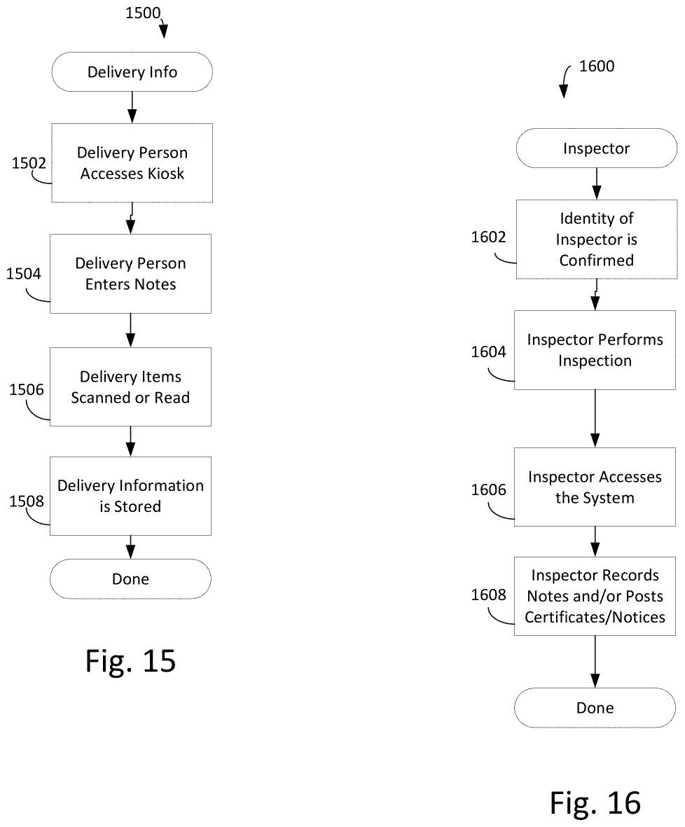

As shown in the diagram of FIG. 15, the materials may be organized into kits for activities at the structure. The material requirements record 2104 is created 2102 and processed to develop the set or group of materials that will be sent to the structure. Different strategies 2108 may deployed to develop the set of materials. For example, the materials in the set may be staged based on the trade involved (e.g., electrical, plumbing, carpentry) so that each trade has its own set for the stage of construction. Thus, sets are selected 2104, and packages are developed for each step or stage of the project or process 2106.

FIG. 16 shows a diagram 2200 of a first example of interactions relating to a smart contract for the project or process. Suppose that the supply company 2202 makes a delivery to the structure. Further suppose that the delivery is confirmed 3308 by information such as that gathered by the system as discussed above. The lender 2204 then releases payment 2210 to the supply company 2202. Payments can be made through third party funding, factoring, credit lines, loans, or other financial option to assist with financing and cash flow management. The smart contract can also credit a payee account and debut a payor account.

The payment may be made electronically, such as through crypto currencies, like Bitcoin or Ether, or via a stable coin whose value is pinned to an item like a paper currency or the like. A cryptocurrency is a digital currency built with cryptographic protocols that make transactions secure and difficult to forge. Other Suitable forms of electronic payment includes Automated Clearing House (ACH) payment, Electronic Funds Transfer (EFT), card payments, other types of bank transfers or other types of electronic wallet transfer. In the case where crypto-currency is used, the crypto-currency may be delivered to the digital wallet of the supply company at a specified wallet address or account 2212. The ledger may be updated to show that the contract is complete 2214. Payment requires that the lender has sufficient funding in their digital wallet 2216. If not, the smart contract will not be written on the immutable ledger 2218. If there is sufficient funding, payment is made, and the contract is written onto the persistence storage as complete at 2220.

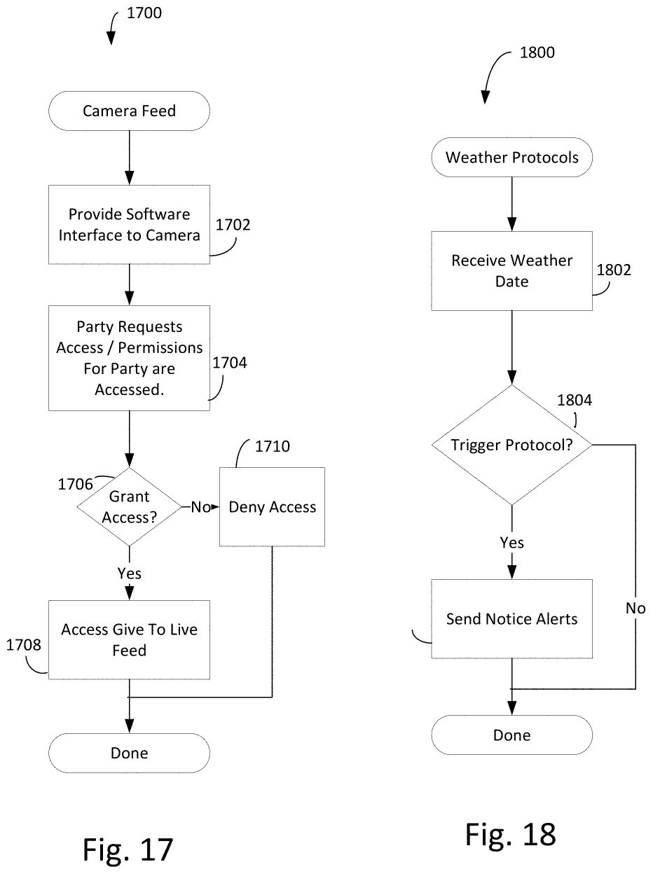

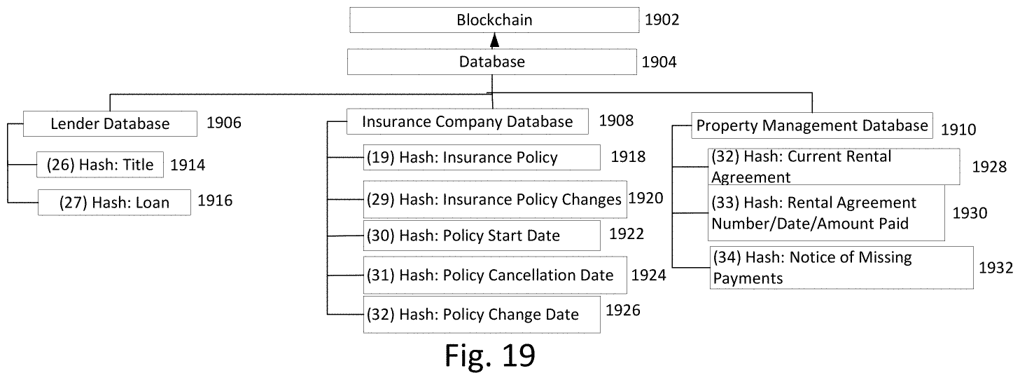

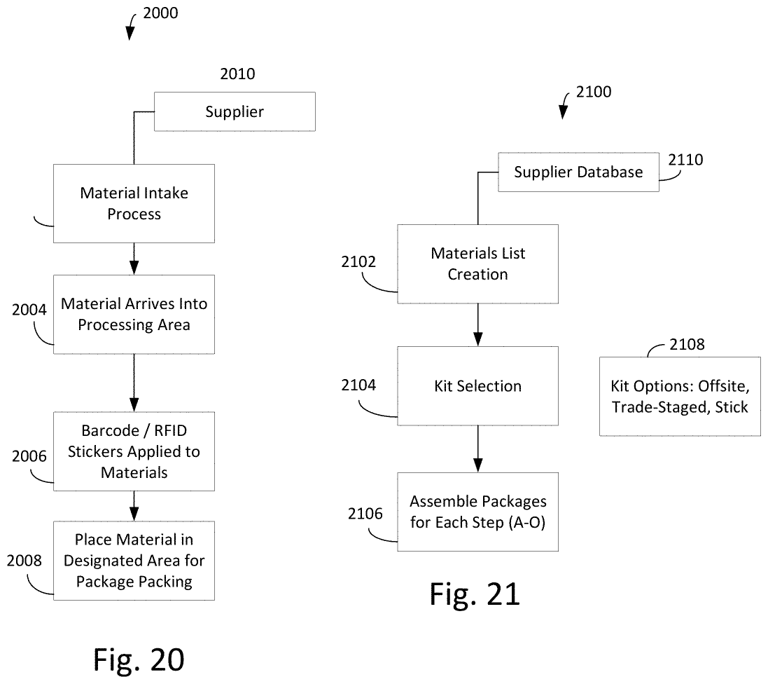

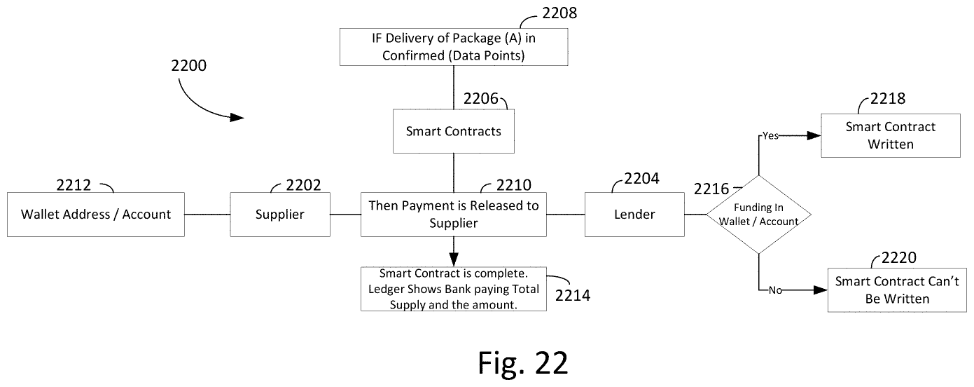

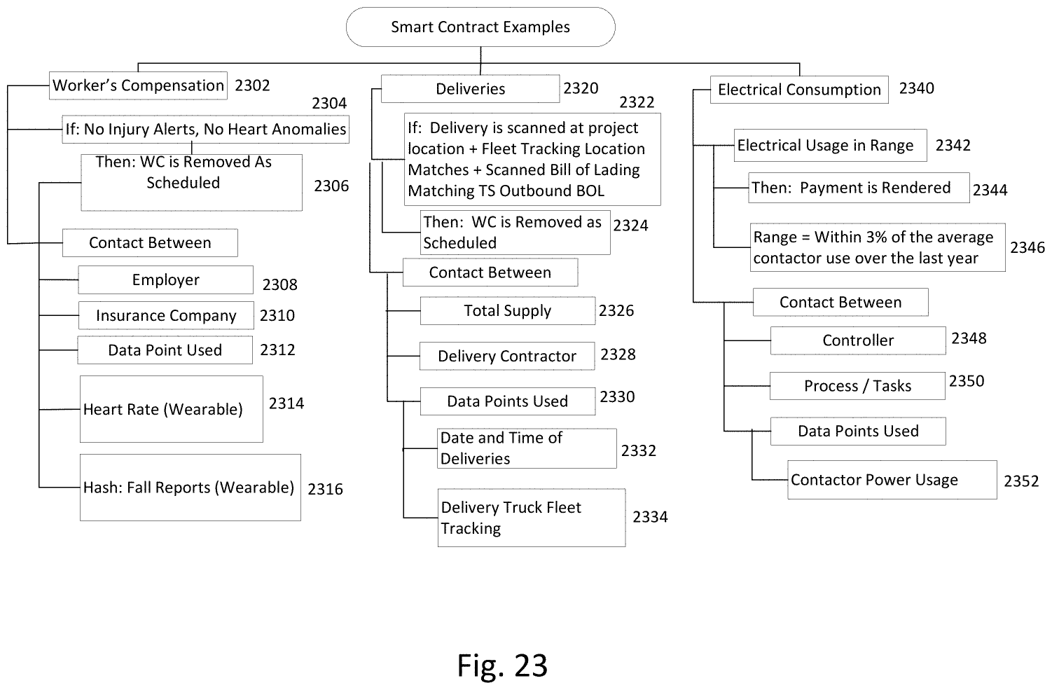

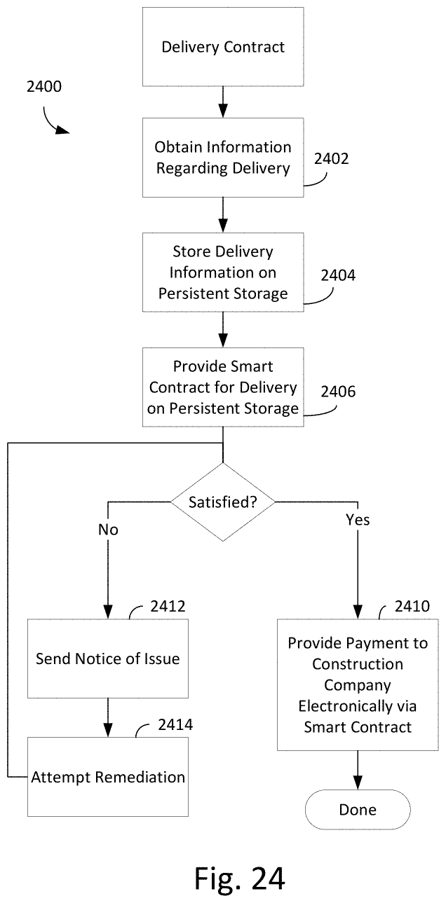

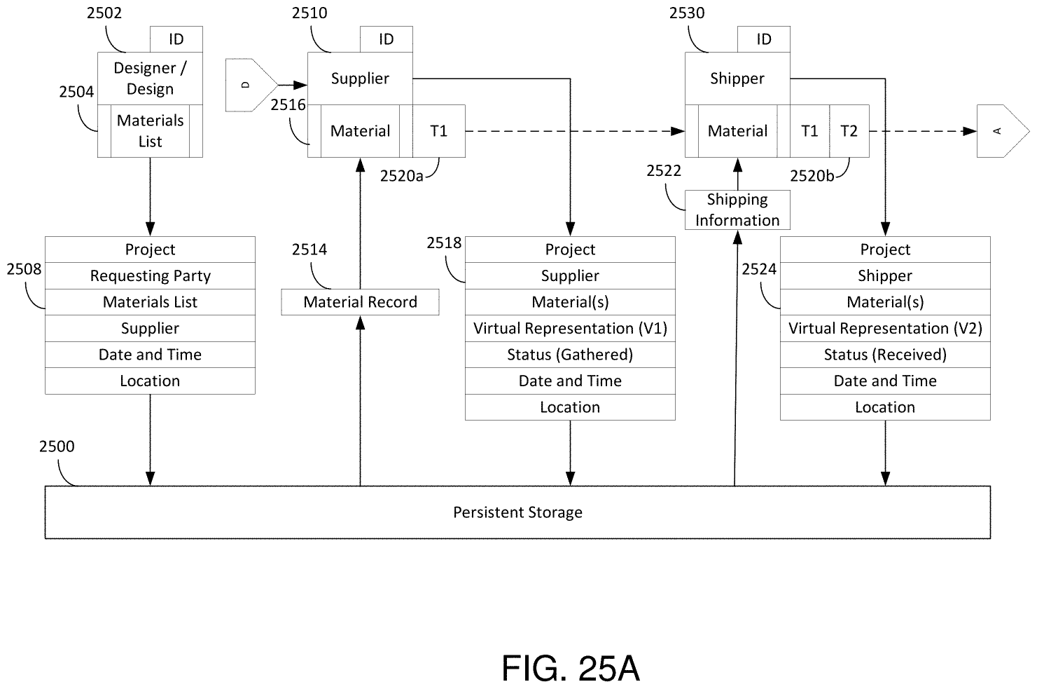

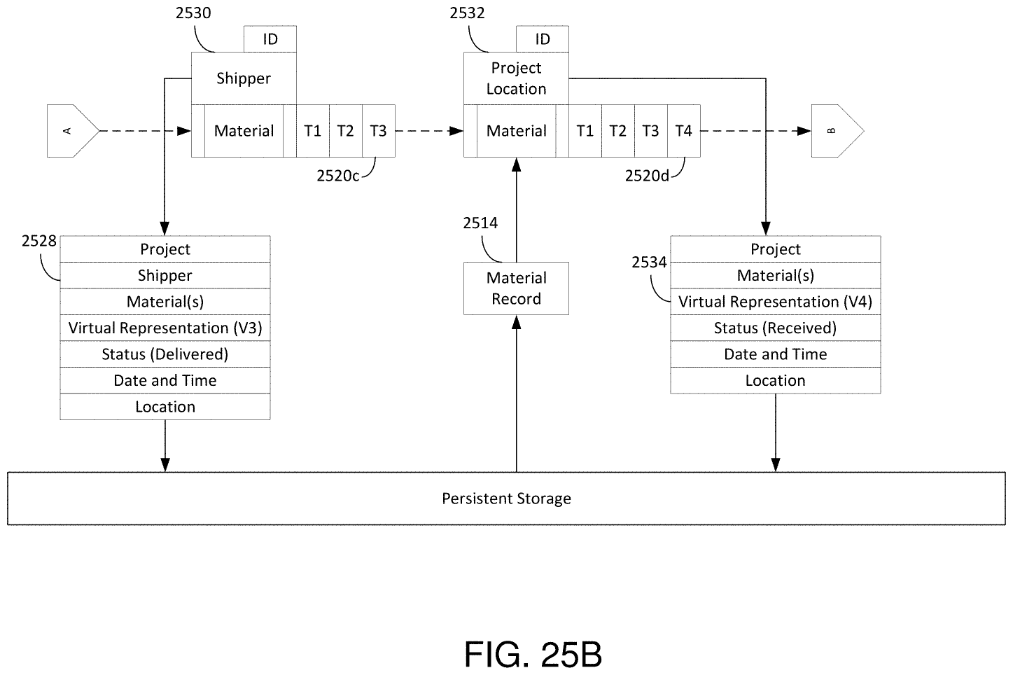

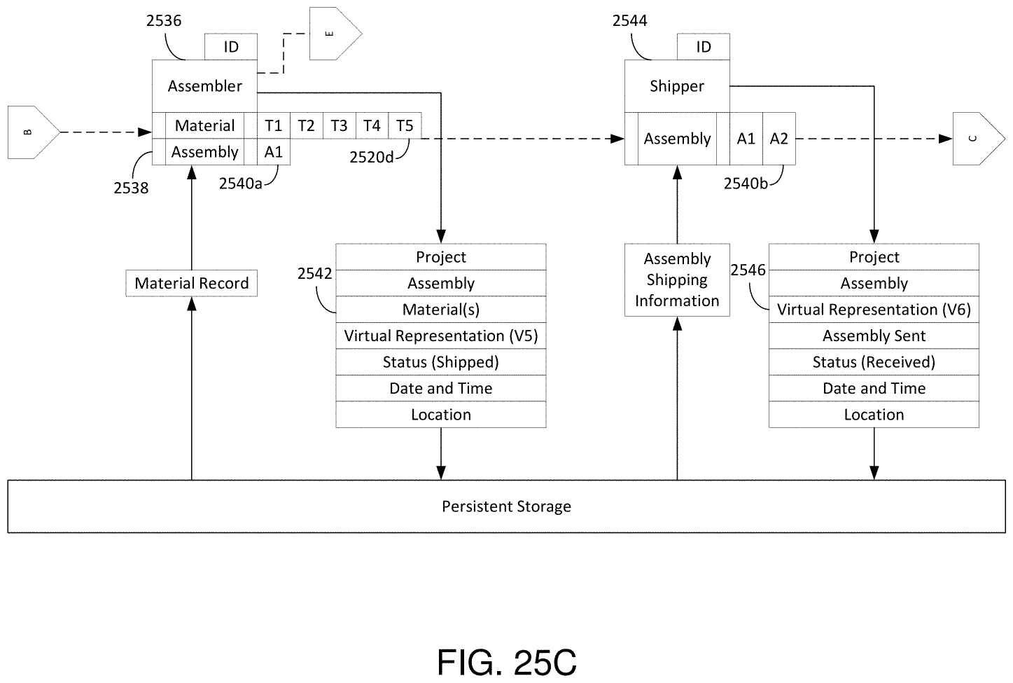

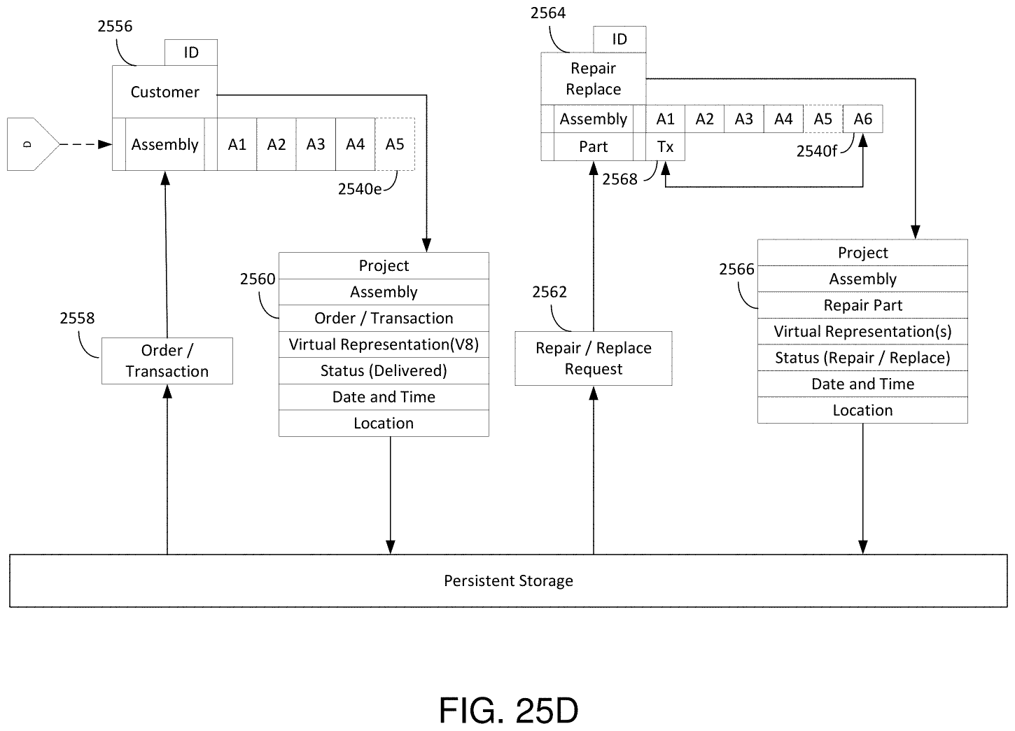

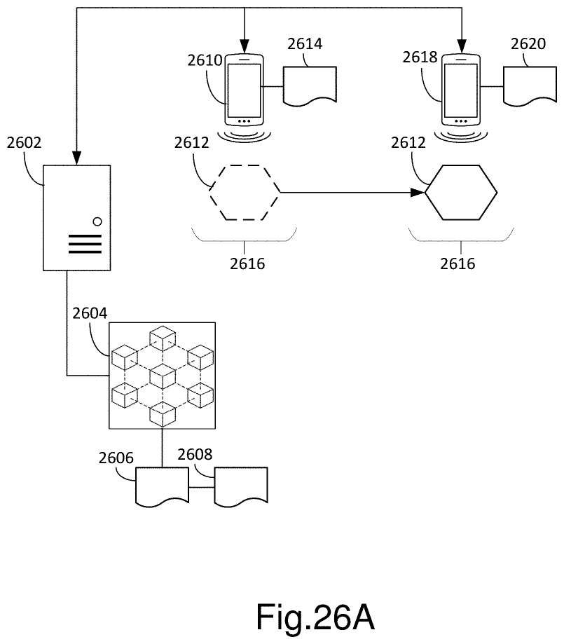

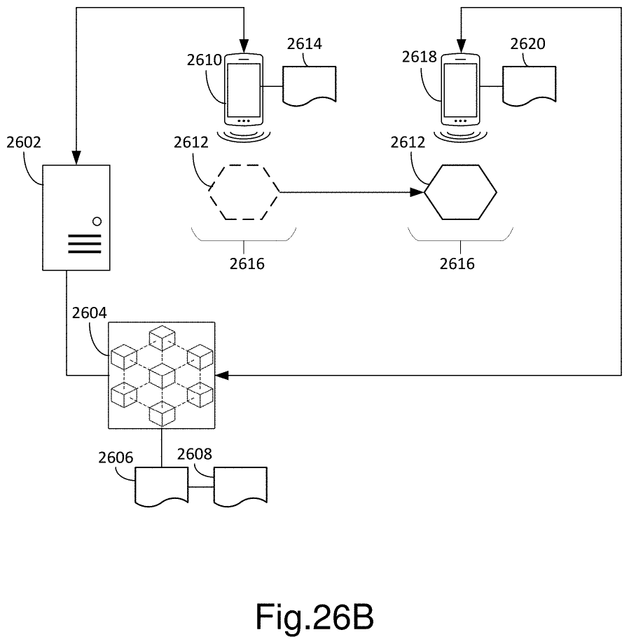

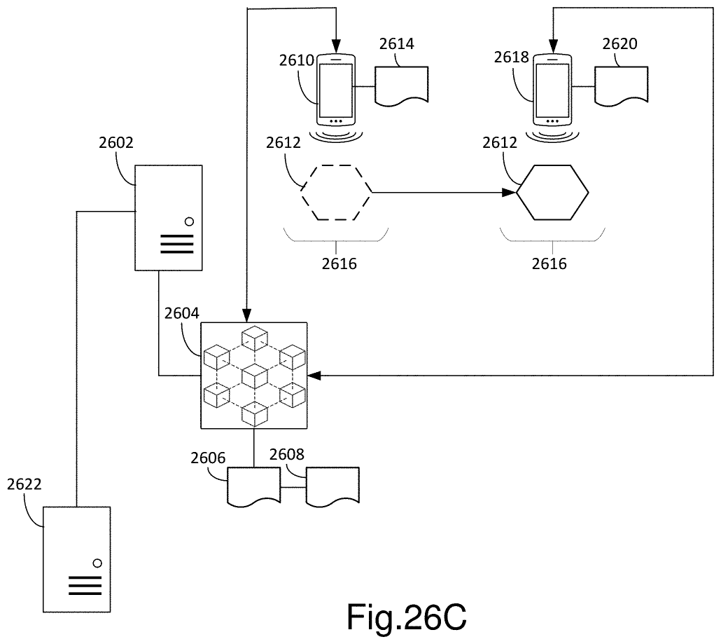

FIG. 17 depicts a diagram 2300 for multiple illustrative smart contracts. In a first illustrative smart contract, the smart contract concerns worker's compensation insurance 2302. The contract removes the insurance for a worker 2306 if there are no injury alerts and no heart rate abnormalities for a given worker 2304. The smart contract can be between entities such as employers (e.g., an assembler) 2308 and an insurance company 2310. The contract looks at the data points 2312 of the heart rate history 2314 gathered by a wearable for the worker and any fall reports 2316 from a wearable for the worker. As mentioned above, the wearables may include a gyroscope or other mechanism that provides data indicative of a fall. This data may be processed to identify data indicative or a fall or other incident where an injury may have occurred.

A second illustrative smart contract shown in FIG. 17 relates to payment for a delivery 2320. If a scan is made at the delivery site, if the fleet location tracking information matches the desired delivery site location and if the scanned material list at the delivery site matches the outbound material list from the supply company 2322, then payment from the supply company 2326 to the delivery worker 2328 is made. Data 2330 used by this illustrative smart contract 2330 includes date and time of deliveries 2332 and delivery truck fleet tracking information 2334.

A third illustrative smart contract shown in FIG. 17 relates to electrical consumption 2340. If the electrical usage by a worker of the assembler is within a range of 3% of the average worker use over the past year 2342 and 2346, then payment is provided 2344 by the assembler 2350 to the system 2348. Power usage data 2352 can be reviewed.

Smart contracts may also play a role with deliveries. First, delivery and/or materials information is obtained regarding delivery to the structure for the project or process 2402. The information obtained can include if the materials delivered match the material requirement record, manufacturer, and/or supplier which can be confirmed by multiple parties.

The delivery information is hashed, and the resulting hash value is referenced on the blockchain-based immutable ledger 2404. A smart contract is provided that uses the immutable ledger 2406. A determination is made whether the conditions specified in the smart contract are satisfied 2408. If the conditions are satisfied, electronic payment for the delivery is realized 2410. If the conditions are not satisfied, notice of outstanding issues are sent and the delivery worker may attempt to remedy the issues 2414. The process may then repeat beginning with step 2408 until the conditions are satisfied.

One of the benefits of the approach adopted by the exemplary embodiments described herein is that a complete record of components deliveries, utilities, transaction and other events associated with the structure is maintained. To pair a material with its virtual representation the system captures events at various points of transition of the material. Pairing the physical material with the virtual representation can include several elements or components. Included in the pairing process can be the physical observation of the physical material and then associate the physical material with a virtual representation so that the physical material is properly associated with the virtual representation. This verification provides trust that the virtual representation is accurately associated with the physical material as a factor rather than simply trusting that the virtual representation is accurate. This system can use manual or automated processes to physically observe the material and associate the material with the virtual representation during various events from raw material to final deliverables. Verification can also use the metadata that is associated with the interaction of physical items by individuals and electronics when the item is created, transported, installed, activated, and destroyed. The metadata that can be captured and placed into immutable ledger can provide stakeholders an audit trail of history for their physical asset using a verified paired virtual representation. A similar process as described herein can be used for pairing a biometric identifier with an individual.

For example, when components are replaced, a replacement part record can be created that captures the replacement process and can include metadata concerning the events and verifications associated with the process. For example, a digital image of the component can be captured, and the images and its metadata of the image captured can be included in the virtual representation. The capture device and its metadata can also be captured and included in the component record. For example, a sensor having a GPS transponder, camera and transceiver can be used to capture events. The metadata of the component (manufacturing, shipping, delivery, installation) can include date, time, location (e.g., GPS coordinates), manufacturing, supply, delivery and installation entity, worker, equipment, and any combination. By capturing the replacement event and verifying that the component and the virtual representation are paired, and stored on the immutable ledger, the physical material and the virtual representation are paired allowing for reliance upon the digital record to accurately represent the physical material.

By verifiably pairing the physical asset with a virtual presentation, the risk of unintentional or impermissible rehypothecation can be reduced or eliminated. The paired asset can be verified by multiparty chronological metadata streams that can be associated with a physical location. Because verifications using these streams are chronological, altering the information could require alteration of the metadata prior to and after the altered record. Therefore, the altered record would be inconsistent with the associated records potentially both temporally and geographically and an attempt to alter the record would be discovered. The use of an immutable ledger further reduces the risk of alterations of records as well as increasing the verification of information. Further, pairing assets associated with the event, involving the asset, interactions with the asset and the associated metadata provide for a substantiated digital asset, reduce, or eliminate risk and improve capital efficiency. Further, the pairing of assets facilitates commerce by allowing electronic transactions with assurances that the virtual representation used in the electronic transaction is paired with the physical asset.

Verification, including verification of an event, can include verifying that the physical material and the virtual representation match and can be accomplished in a variation of methods including interaction with identification elements such as a tag, label, and the like, capturing an image of the material, capturing a video of the material, capturing a tag physically affixed or otherwise associated with the material, human visual inspection, and any combination. Identification of an individual performing or otherwise associated with an event can be captured by identification devices (e.g., cards, tags, RF ID) and biometrics including visual capture (e.g., facial recognition), voice recognition, iris scan, fingerprint, palm print and any combination.

The system can retrieve the component record, receive verification that the component delivered to a structure is the same that was requested and create or update an associated record. A verification that the component and the retrieved virtual representation match can be performed using a capture device, worker verification and a combination. The metadata associated with delivering the component to the shipper can be captured and included in the shipping record. The shipping record can include information about the shipper and the worker delivering the component to the shipper. The shipping record can include information about the destination of the component. By capturing the shipping event and verifying that the component delivered to the shipper and the virtual representation are paired, and stored on the immutable ledger, the physical material and the virtual representation are paired from manufacturing of the component to the delivery to the shipper allowing for reliance upon the digital record to accurately represent the physical material and its disposition.

The system can retrieve the shipping record, receive verification that the component delivered by the shipper to a structure is the same that was manufactured, requested, suitable for the structure, shipped, and received. The metadata associated with delivering the component to the structure can be captured and included in a component or replacement part record. The component or replacement part record can include information about the shipper, processor, worker and any combination. The record can include information about the processor, location, and other information.

The immutable ledger 2500 is accessible by an owner or other computer system 2502. The requesting party can have a unique ID associated with it. The requesting party can create a maintenance record such as a replacement part, project, process, or other activity. For example, the project can include a replacement part, a repair, new constructions, payment, or utilities, tracking utilities, or other activity. The project can include a material list that will be used for the project. The system can create an event designer record 2508 that can include information associated with the project, structure, designer, material list, supplier for one or more materials on the material list, the date and time the design was created or modified and the location where the design was created or modified and other metadata. The design record can be stored on the immutable ledger that can be local or remote from the designer.

For example, if the project is a new construction project, the structure would be the physical location of the construction site. The material list can include a material requirement record which can be generated by a designer as well as integrators, service providers, resellers, and others and can be sent to suppliers or manufacturers so that the identification and cost of the material requirement and task to be performed with the materials are known prior to ordering the materials and finalizing a project plan or process. The material record can be stored on the immutable ledger. The material requirement record can include a single component or multiple components. Each component or material in a set of materials can include a warranty that is from the manufacturer, distributor, installer, and any combination. The requirements for a warranty to remain in place can include requirements such as compliance with installation processes, environmental conditions, use of licensed workers, use of qualified and experienced workers and any combination.

A supplier, using a supplier computer system 2510, can select or otherwise acquire the material 2516 identified on the material list from a materials requirement record 2512 or designer record that can be retrieved or otherwise received by the supplier computer system from the immutable ledger. The supplier can verify that the material matches the material requirement record, and the system can capture this event. For example, one method of associating the physical material with a virtual representation is using a tag 2520a (T₁) placed on the material. The tag is then physically verified to be associated with the material from the material list or the material requirement record. Therefore, the physical material and the virtual representation (V₁) are paired by recording this event and associating the physical material, T₁, and V₁. In one embodiment, the tag can include the following information:

| Description | Digits | Information |
|---|---|---|
| Locations | 19-20 | GPS XX.XXXXXX XXX.XXXXXX |
| User ID | 8 | SSN XXXX + Initials XX + Gender X |
| Date | 10 | XX/XX/XXXX |
| Time | 7 | Zulu XXXX:XX |
| Material | 12 | UPC/Barcode XXXXXXXXXXXX |

A supplier record 2518 can be created and stored on the immutable ledger. The capture event can include a unique number and include the supplier ID, date and time, location, material ID, status, and any combination. The material ID can be from an original manufacturer or the supplier. The status can include that the material has been gathered, packaged, ordered, is in stock or on back order, shipping information and any combination. The shipping information can include the origin, destination, shipping instructions, shipper, and any combination.

The shipper can verify that the physical materials being retrieved from the supplier match the virtual representation of the maintenance record, maintenance request, component history, structure history or audit trail. If the components are verified, the shipper can physically capture the event, for example, by affixing its tag 2520b (T₂) to the materials representing this verification. A supplier shipping pickup record 2524 can be created and stored on the immutable ledger. The supplier shipping pickup record can include project, shipper, material, status, date, time, location, and any combination. The mode of transportation of the material can also be tracked and stored on the immutable ledger. For example, if the shipper uses a vehicle, the date, time, location, and other metadata associated with the vehicle can be gathered along the route and stored on the immutable ledger. Verification can be provided using the metadata of the various events. For example, if the date, time, and location of the supplier record is within a certain range of values of the date, time and location of the supplier shipping pickup record, there will be verification that the proper materials were physically transmitted from the supplier to the shipper.

The shipper can deliver the material to the destination such as a structure. When the shipper delivers the materials to the structure, the shipper can capture this event by creating a supplier shipping delivery 2528 record using a shipper computer system 2530. The shipper can verify the event by methods including adding a tag 2520c (T₃) representing that the proper materials were delivered to the proper location. The structure computer system 2532 can be used to verify that the materials were properly delivered by retrieving the material record 2514 from the immutable ledger and using the record to match the physical materials delivered. In one embodiment, the shipper can use the tags that are part of the virtual representation to match T₃ with the material and the information stored on the immutable ledger to capture and verify the event. When the material is delivered, the structure can use a project computer system 2532 to retrieve the material record from the immutable ledger and match the material delivered with the material record. The structure can add a tag 2520d (T₄) to the material to capture this event. The structure can create a structure material received record 2534 that can include the project, material, virtual representation (V₄), status, date, time, location, other metadata, and any combination. The shipper, worker at the structure, or both can physically inspect the material and verify that it is matches the virtual representation stored on the immutable ledger. This verification can be included in the information that is stored on the immutable ledger by the shipper and a worker or system at the structure.

Further a sub-project can be treated as a project as discussed herein.

A controller 2536 can be used to retrieve the component record or maintenance request record from the immutable ledger. The component record can be used to match the component delivered to the structure to verify that the proper component was received at the structure. The receiving party can add tag 2520d (T₅) to the material, or use other verification methods described herein, to capture the event. The receiving party can also capture the component used at 2538 by adding a tag 2540a (A₁) to the assembly. A component record 2542 can be created and stored on the immutable ledger. The component record can include the project, assembly description and other information, assembler, material(s) used, virtual representation, shipping information date, time, location of the assembly, other metadata, and any combination.

Once completed, and if there are intermediate steps in a repair, the component may need to be delivered to another location. The component record can include shipping information, or a component shipping record can be created and stored on the immutable ledger. If the component needs to be delivered, a second shipper can use a second shipper computer system 2544 to retrieve the shipping record, assembler record or other shipping information that is used to identify the origin, locations, assembly, pick up time, delivery time and other information associated with the transportation of the assembly from one location to another. The assembly can be received by the second shipper and the second shipper can capture the event such as with a tag 2540b (A₂) to the assembly representing that the assembly has been verified by the second shipper as properly provided and received by the shipper. A second shipper pick up record 2546 can be created and stored on the immutable ledger.

The customer can be shipped the assembly using the system as described herein with a shipper performing the steps and the system performing the steps associated with the shipper and second shipper above. A third shipper can create a third shipper pickup and delivery record that can be stored on the immutable ledger verifying that the assembly was properly provided from the realtor or distributer to the customer. The customer may capture the event and can add a tag 2540e (A₅) to the assembly that can be associated with the virtual representation (V₈). A customer record 2560 can be stored on the immutable ledger.

Using this system, the customer can be assured that the assembly was independently verified and authenticated from the design to the delivery to the customer and that the virtual representation of the assembly and its components (e.g., materials) are paired.

In one embodiment, a repair request 2562 can be created and stored on the persistent server. The repair request can be associated with the assembly and retrieved by a repair computer system 2564. The repair company can receive a part using the system described herein, perform a repair or replacement action, and capture the event such as by using a tag 2540f (A₆) to the assembly indicating that the assembly has had a part repaired or replaced. The repair part can also have a preexisting tag from the use of the system herein and the repair company can capture the event such as by using a tag 2568 (Tₓ). A repair record 2566 can be created and stored on the immutable ledger.

The system described herein can pair the physical material and/or assembly with a virtual representation. Failure to pair the physical material or assembly with the virtual representation can negatively impact areas such as regulatory requirements. Regulatory requirements are a set of rules that can specify the standards for a project. Regulatory requirements impact designs, materials, worker's license and experience the project and process. For example, a building code may require that construction materials be installed in accordance with manufacturer's specifications and warranty regulations. Failure to follow the building codes can result in the project not being approved, errors, lack of customer satisfaction, insurance claims, injury, litigation, and other negative ramifications. Tracking, management, and verification of materials to ensure compliance with regulatory requirements and proper installation according to applicable specifications is an important aspect to many projects and processes. Tracking and record keeping during the project or process can be beneficial, as it can be difficult to perform these tasks after project or process completion because the materials can be hidden from view or otherwise inaccessible. For example, electrical wiring in a project or process can be hidden behind walls and ceilings once the project is complete.

Automated verification of quantities, quality, and correct product deliveries along with after delivery tracking of materials with accountability is seldom used. Designated delivery areas with geofenced control and tracking of materials once delivered would help prevent loss. Confirmation of products integrated at the asset location provides transparency regarding sourcing, warranties, as well as future reference during the structure and individual product's life of use. By using the various tags and virtual representations, each entity in the process can verify that the physical materials match any record the precedes that entity.

This process can include internal and external individuals and machines for performing inspections (e.g., verifications). For example, the system can receive a set of internal inspection information entered into the system from an internal inspector representing an internal physical inspection of the project, material or assembly. As the items travel, an internal inspector can provide inspection information representing the stages of the project. The system can also receive a set of external inspection information from an external inspector and an external inspection computer device representing a third-party physical inspection of the project at predetermined stages of the project. Based upon the internal inspection, external inspection or both, an inspection record can be created and stored on the immutable ledger.

A computer system 2602 can be in communications with a data storage system 2604. The data storage system can be permanent, immutable, and persistent so that the information stored on the data system, once storage, cannot be changed. The data system can include a plurality of computer systems where data can have copies onto each computer system. When using the data storage system, the data can be static so that once created and stored, it cannot be changed. Examples of data storage platforms include hard drives, solid state drives, tapes, and cloud storage systems. The immutable data storage system can use blockchain, crypto-shredding, WORM, append only, distributed ledger technology, immutable cloud storage, immutable record retention such as Oracle Cloud Infrastructure Object Storage, and any combination thereof. In one embodiment, the immutability is accomplished by the data storage system only allowing records to be appended to the storage media without the ability to modify the record one written. One such system includes blockchain. When a first record 2606 is written to the data storage system, the record cannot be changed. When a second record 2608 is written to the data storage system, it is stored later in time to the first record thereby effectively providing a chronologically trail of events associated with the individual and the digital representation of the individual and activity. In one embodiment, the second record can include information linking it to the first record including a hash from the first record.

In one embodiment, the first record can be associated with a first event and a second record can be associated with a second event. Each record can include biometric data of an individual associated with each event. The individual can be the same individual for each event of difference individuals for each event. These events can be transactions, processes, or other activities. The order the first record and the second record can be written on the immutable storage can be used to show that some period of time elapsed between the first record and the second record. This functionality can add to the verification process and attempts to improperly tamper with the immutable storage may be discovered when the first record and the second record are not in chronological order. Further attempts to improperly tamper with the immutable storage can be discovered when the metadata of the first record and the second record are inconsistent with the first record and the second record being stored chronologically. For examples of the metadata of the first event includes a first time and first location and the meta data of the second event include a second time and second location where it is physically impossible for the individual associated with the two events to physically move between the first location and the second location in the time between the first time and the second time.

The first event record can be associated with a first event and the second event record can be associated with a second event. The data associated with these events can be retrieved from a capture device and used in creating and writing the associated event record. The data captured can include biometric data of an individual associated with one or more events. If the date and time setting of the data capture device are incorrect, this discrepancy can be identified by comparison with the event record created and compared to related records in the immutable storage. In one embodiment, metadata integrity used by the system can be designed to identify inconsistencies with date and time. The first event can differ from the second event by time, activity, process, location, or any combination.

In one embodiment, metadata associated with the event and event record and a ledger hash time, representing when the event record is stored (e.g., committed) to the immutable storage can be used to validate the metadata provided from the data capture device. If the data capture device has an incorrect time, a comparison of the ledger hash time with the metadata from the data capture device can identify an error. Identifying an error can be used to alert users to data capture device issues and can indicate that the data capture device needs to be serviced or replaced prior to its next use. The data capture device can include a biometric reader or scanner. The metadata captured from the data capture device can include the device identification, model, manufacturer information, error codes, status information, user data, environmental data (e.g., weather data), software information, hardware information, date field information, and the like.

The metadata can include biometric data that may include facial recognition, an iris/retinal scan, a fingerprint scan, a hand scan, a voice print, or heart rate signature and any combination. The metadata that can be associated with the capture device can include weather conditions, which can include a sun angle, which can be compared with environmental weather conditions to approximate the data capture time. Metadata associated with an image or video can be used to verify weather conditions in the image or video. Metadata associated with an image or video can be used to verify biometric data of the individual.

For example, a camera can capture an image of an individual. The metadata (e.g., a first metadata) associated with this image can include the date, time, locations, device number and other data. The image can include in the background the shadow of the individual or a shadow of another object. Weather information (e.g., a second metadata) can be accessed from a third-party source such as the national weather center or standard organization such as the National Institute of Standards and Technology and the National Oceanic and Atmospheric Administration of the United States Department of Commerce. One verification can include that the image shows that at the captured locations, date and time, there was sunlight. The weather data can indicate if it was sunny. If these two conditions exist, the confidence that the individual was at the captured location and the date and time that the individual was at that location is increased and included in the verification.

The system can also retrieve the previous capture location, date and time (e.g., a third metadata) of the individual and compare it to the currently captured location date and time. A determination can be made concerning the time and speed it would take to travel from the previous location to the current location. For example, the previous location is thirty miles from the current location and the time difference between the previous data capture and the current data capture is ten minutes, confidence that the current data capture is accurate is reduced.

For example, the video can use facial recognition to verify that the individual entering a fingerprint is the same individual recognized on the image. Time and location metadata can be retrieved from publicly sources or remote sources and captured with the device metadata to determine of the captured weather in the image or video is the same as being reported locally on that day and at that time.

The metadata that is captured can be dependent upon the device and can include metadata associated with an individual (e.g., worker), equipment, weather, enterprise software, security hardware and software, material, indicia, smart contracts, public records, authentication information, date, time, location, entity, and any combination of these examples.

In one embodiment, an image or video captured can be used to identify an approximate time where data, including biometric data, was captured by the data capture device. The metadata associated with the data capture can include weather conditions, sun angle, which can be compared with environmental weather conditions to approximate the data capture time. In one embodiment, the data capture can include the location so that the location of the data capture device can be used to retrieve environmental weather conditions when the data capture occurs. In one embodiment, the system can determine the length of a shadow and according to the position of the capture device, the time of day can be determined. Using this information, the confidence of the accuracy of the time of the capture device can be increased. This information can also be used to increase the confidence in the accurate of the capture device reported location as the shadow length should be consistent with the location, object height and time.

The data capture device can capture data in response to an event associated with the physical object. For example, if the physical object's location changes, is modified, transferred, integrated, or other action, process or procedure associated with the physical object can signify an event. A location can include a manufacturing place, construction site, business providing services (e.g., vehicle repair service), origination site, delivery site or other location where the materials will be used including the creation, maintenance, repair, or integration into an assembly. Further, if an individual interacting or otherwise associated with a physical object changes, such data can be captured.

Verification, including verification of an event, can include verifying that the individual information and the virtual representation match and can be accomplished in a variation of methods including capturing biometric information, capturing an image of video, capturing indicia such as a card, fob, of another item otherwise associated with the individual. The identification of an individual performing or otherwise associated with an event can be captured by identification devices (e.g., cards, tags, RF ID) and biometrics including visual capture (e.g., facial recognition), voice recognition, iris scan, fingerprint, palm print, weight, dimensions, change in weight, dimensions or other attributes, and any combination. Examples of verification processes can include having stored data about the physical object and comparing the physical object with the date, using machine learning process video, using imagery, audio clips and other media to and any combination. Individuals, such as inspectors or verifiers, can be used to verify physical objects and events onsite and offsite. Individuals can process video, imagery, audio clips and other media to verify assets and events and provide the verification to the system at one or more events. Upon verification of an event, smart contracts can be executed according to verification of the physical object and event. The individual preforming these activities can be identified through the capture of biometric data. The system can also retrieve event records that can include data and metadata about an individual, including biometric data. The data and metadata from a first record can be compared with a second record or subsequent record to determine if the individual represented by the first record is the same as the individual represented by the second or subsequent record.

In operation, a first data capture device 2610 can be in communications with the computer system 2602 so that data captured by the first data capture device can be transmitted to the computer system. The first data capture device can have a first capture device metadata 2614 originating from the data device that can be included in the first record 2606. Capture device metadata can include the biometric data of the individual associated with an event. The first data capture device can capture data associated with the physical object and the individual. Object data can include an image of the physical object or individual, tag, label, RFID, weight, dimensions, and other indicia and any combination thereof. The object and individual data can be captured at a first event 2616 that can include a change in state of the physical object, change in location change in time or any combination thereof. When an event occurs, which can be a second event, a second data capture device 2618 can have a second capture device metadata 2620 originating from the data device that can be included in the second record 2608. The second data capture device can also capture object and individual data associated with the physical object and individual 2612 at the second event 2620.

During data capture by the first data capture device, indicia can be capture where the indicia is associated with the object or individual. The indicia can include biometric data, an image, a label affixed to the object, a radio frequency identification (RFID) tag, an ultra-high frequency (UHF) tag, a bar code, a QR code, a Bluetooth beacons, alpha-numeric characters, and any combination thereof. The indicia can be included in the first event record and stored on the immutable storage. When a change in time, location or other event occurs the second data capture device can capture the indicia. Once captured, the indicia can be compared to the indicia in the first event record and if the two matches, then verification exists that the individual associated with the second event is the same individual that was present at the first event. In one embodiment, the second capture device can capture data, transmit the data to the computer system 2602 and computer readable instructions on the computer system can perform the comparison of the indicia capture as the second event with the indicia included in the first event record.

The second data capture device can be in communications with the immutable storage. Computer readable instructions on the second data capture device can capture the data at the second event, retrieve the first event record, compare the indicia from the second capture device with the indicia of the first event record and determine if the physical at the second event is the same physical object at the first event. In one embodiment, the second data capture device can store a second event record that can include indicia capture at the second event on the immutable storage. The computer system can be notified that a second event record has been stored. The computer system can retrieve the first event record and the second event record and compare the respective object indica to determine of the physical object is the same physical object at the first event and the second event. If the object indicium is not the same, a notification can be provided indicating that the physical object has been changed, modified or otherwise different between the first event and the second event.

The first data capture device can be in communication with the immutable storage. In one embodiment, the first data capture device can capture data at a first event that can include indicia, create a first event record, and store the first event record on the immutable storage. The second data capture device can retrieve the first record having the indicia from the immutable storage and compare the indicia captured by the second data capture device with the indicia of the retrieved first event record. The first data capture device and the second data capture device can be the same device.

The system can therefore pair an individual with a digital representation, such as an indicium, and verify among events that the individual has not changed. The system can also determine if the biometric data of the individual capture during an activity is the same data or represents the same individual in the previous of subsequent record. The system can also determine if the biometric data of the individual capture during an activity is the same different data or represents a different individual from the previous or subsequent record.

The data capture devices can serve as data capture nodes for a system where the data capture devices are distributed. The data and metadata that is capture from the data capture devices can be transmitted directly or indirectly to the immutable storage. The stored data and metadata can be configured to provide for an immutable digital audit trail wherein each metadata stored can include time, location, and activity of the individual and physical objects. When needed, the stored data and metadata can be accessed and using the time, location, activity, individual or physical object, linked together to form the immutable digital audit trail. Multiple audit trails can be provided according to time, location, activity, individual, physical object and any combination. For example, if there is an inquire as to the status of a delivery, the system access the data and metadata and retrieve the data and metadata associated with a physical object, arrange the data and metadata in coprological order, and determine the time and location that the physical object was present to assist with tracking the physical object and the individual associated with the physical object (e.g., delivery worker, assembler, retail seller and the like).

In one embodiment, a first data capture device can be in communications with a second data capture device so that the data and metadata from the first data capture device can be compared to the data and metadata of the second data capture device to provide a level of verification of the data and metadata captured by each device. For example, if the first data capture device and the second data capture device both capture the same individual biometric data within a predetermined period of time and the distance between the first data capture device and the second data capture device can be traveled in the predeterminer period of time, the confidence that the individual was at both locations at the time indicated is increased. Further, the first data capture device can be a kiosk and can be in communication with the second data capture device.

The system can include one or more application programming interfaces (API) allowing the system to communication with third party systems and allowing such third-party systems to benefit from the functionality of the present system. For example, a third-party system can be used for lease management. By accessing the data of this the third-party leasing system, the present system can receive lease information, payment information, occupant information and the like. The present system can them pair the information received from the third-party system and verify that the digital information from the third-party system is accurately associated with the physical structure, transaction, occupant, owner of other physical item or individual. Therefore, the present system allows for pairing of digital representation taken from third party system with the associated physical item or individual to provide truth over trust.

While the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the following claims, including ail modifications and equivalents.

What is claimed is:

1. A computerized system for pairing physical objects with virtual representations comprising:
a structure;
a controller carried by the structure, uniquely associated with the structure and in communication with an immutable ledger, a remote delivery computer system, a payment management computer system and a remote maintenance computer device;
an input assembly in communications with the controller;
a set of computer readable instructions included in the controller adapted for:
retrieving a payment account from the payment management computer system wherein the payment account is associated with the structure wherein the payment account is selected from the group consisting of a mortgage, lease, rent, utility, insurance, tax, common area dues, homeowner dues, regime fees, and any combination thereof, initiating a payment according to the payment account, storing a payment record associated with the payment on the immutable ledger, and,
receiving a maintenance request from the input assembly, determining a component from the maintenance request, retrieving a component history from the immutable ledger, determining a replacement part from the component history, initiating a delivery of the replacement part to the structure, verifying that the replacement part delivered is consistent with the component history using the input assembly, determining a repair service from the maintenance request and component history, dispatching an approved service technician according to the repair service to the structure, receiving a repair status from the input assembly, storing the repair status on the immutable ledger thereby updating a digital audit trail associated with the structure, initiating a maintenance smart contract.

2. The computerized system of claim 1 wherein the set of computer readable instructions adapted for initiating a payment includes transmitting a payment request to a payor computer system representing a debit of a payor account and a credit of a supplier account.

3. The computerized system of claim 2 wherein the payor account is associated with an occupant of the structure.

4. The computerized system of claim 2 wherein the payment management computer system is adapted to receive third party credit for use in crediting the supplier account.

5. The computerized system of claim 1 wherein the set of computer readable instructions adapted for determining a component from the maintenance request includes retrieving a bill of materials from the immutable ledger associated with the structure.

6. The computerized system of claim 5 wherein the bill of materials is stored on the immutable ledger by a kiosk during construction of the structure.

7. The computerized system of claim 5 wherein the set of computer readable instructions adapted for determining a replacement part includes retrieving a final bill of material from the immutable ledger and requesting a substitute part from a supply company computer system.

8. The computerized system of claim 1 wherein the set of computer readable instructions adapted for verifying that the replacement part delivered is consistent with the component history using the input assembly includes detecting the replacement part at the structure using a first sensor and a second sensor included in the input assembly.

9. The computerized system of claim 8 wherein the set of computer readable instructions adapted for verifying that the replacement part delivered is consistent with the component history using the input assembly includes determining if a first data set received from the first sensor is consistent with a second data set received from the second sensor.

10. The computerized system of claim 1 wherein the set of computer readable instructions adapted for receiving a repair status from the input assembly includes receiving biometric data from a repair worker disposed at the structure.

11. The computerized system of claim 10 wherein the set of computer readable instructions adapted for receiving biometric data includes verifying that the repair worker is an approved worker according to the maintenance request and the replacement part.

12. The computerized system of claim 1 wherein the set of computer readable instructions adapted for receiving a repair status from the input assembly includes receiving an installation status from the input assembly.

13. A computerized system for pairing physical objects with virtual representations comprising:
a structure;
a controller uniquely associated with the structure and in communication with an immutable ledger;
an input assembly in communications with the controller;
a set of computer readable instructions included in the controller adapted for receiving a maintenance request from the input assembly identifying a component, retrieving a component history from the immutable ledger, determining a replacement part from the component history, initiating a delivery of the replacement part to the structure, verifying that the replacement part delivered is consistent with the component history and a warranty using the input assembly, detecting the replacement part at the structure using the input assembly, storing a replacement part record in the immutable ledger thereby updating a digital audit trail associated with the structure.

14. The computerized system of claim 13 wherein the set of computer readable instructions adapted for retrieving a warranty record from the immutable ledger and verifying that the replacement part is consistent with the warranty record.

15. The computerized system of claim 13 wherein the set of computer readable instructions adapted for detecting the replacement part at the structure using the input assembly includes detecting the replacement part using a first sensor associated with a delivery worker computer device and a second sensor associated with the input assembly.

16. The computerized system of claim 13 wherein the set of computer readable instructions are adapted for determining a repair service from the maintenance request and component history, dispatching a service technician to the structure, receiving a repair status from the input assembly, and storing the repair status on the immutable ledger.

17. The computerized system of claim 13 the set of computer readable instructions for receiving a repair status from the input assembly includes recording video information of a performed repair.

18. The computerized system of claim 13 wherein the set of computer readable instructions are adapted for receiving a repair status from the input assembly, storing the repair status on the immutable ledger thereby updating a digital audit trail associated with the structure, initiating a maintenance smart contract according to the repair status.

19. The computerized system of claim 20 wherein the repair status is verified by a third-party computer system.

20. A computerized system for pairing physical objects with virtual representations comprising:
a structure having a static location and associated with an occupant wherein the occupant has an occupant identification.
a controller uniquely associated with the structure and in communication with an immutable ledger, a remote utility computer system, a remote delivery computer system, and a remote maintenance computer device.
an input assembly in communications with the controller.
a set of computer readable instructions included in the controller adapted for receiving an occupant identification from the input assembly, associating the occupant identification with the structure, retrieving a reported utility usage from the remote utility computer system, recording a structure utility usage from the structure, determining if the reported utility usage is consistent with the structure utility usage and if so, initiating a utility payment according to the reported utility usage, storing a utility payment record associated with the occupant and the structure on the immutable ledger.

21. A computerized system for pairing physical objects with virtual representations comprising:
a structure having a static location and associated with an occupant wherein the occupant has an occupant identification.
a controller uniquely associated with the structure and in communication with an immutable ledger, a remote utility computer system, a remote delivery computer system, and a remote maintenance computer device.
an input assembly in communications with the controller; and,
a set of computer readable instructions included in the controller adapted for retrieving an occupant payment account from the immutable ledger, initiating an occupancy payment according to the occupant payment account, storing an occupancy payment record associated with the occupant and the structure on the immutable ledger thereby creating a payment audit trail associated with the occupant and the structure.

22. The computerized system of claim 21 wherein the set of computer readable instructions adapted for initiating an occupancy payment includes transmitting an occupancy payment request representing a debit of an occupant account and a credit of a management account.

23. The computerized system of claim 21 wherein the occupant payment account is taken from the group consisting of mortgage, lease, utility, insurance, tax, common area dues, homeowner dues, regime fees, and any combination thereof.

24. The computerized system of claim 23 where the set of computer readable instructions are adapted for receiving an occupant identification from the input assembly, associating the occupant identification with the structure, retrieving a reported utility usage from the remote utility computer system, recording a structure utility usage from the structure, determining if the reported utility usage is consistent with the structure utility usage and if so, initiating a utility payment according to the reported utility usage, storing a utility payment record associated with an occupant and the structure on the immutable ledger.
